(19) United States
(12) Patent Application Publication
(10) Pub. No.: US 2018/0155411 A1
(43) Pub. Date: Jun. 7, 2018

(54) HETEROTYPIC ANTIBODIES SPECIFIC FOR HUMAN ROTAVIRUS

(71) Applicant: The Board of Trustees of the Leland Stanford Junior University, Stanford, CA (US)

(72) Inventors: Nitya Nair, Oakland, CA (US); Lisa Blum, Mountain View, CA (US); Ningguo Feng, Cupertino, CA (US); William H. Robinson, Palo Alto, CA (US); Harry Greenberg, Palo Alto, CA (US); Mrinmoy Sanyal, Palo Alto, CA (US)

(21) Appl. No.: 15/737,634

(22) PCT Filed: Jul. 8, 2016
(86) PCT No.: PCT/US2016/041613

§ 371 Date: Dec. 18, 2017

Related U.S. Application Data
(60) Provisional application No. 62/189,901, filed on Jul. 8, 2015.

(51) Int. Cl. C07K 16/10; G01N 33/569; C12Q 1/6869; A61P 31/14

(52) U.S. Cl. CPC C07K 16/10; G01N 33/56983; C12Q 1/6869; A61P 31/14; C07K 2317/56; C07K 2317/21; C07K 2317/622; C07K 2317/76; C07K 2317/33; C07K 2317/565

(57) ABSTRACT

Compositions and methods are provided relating to rotavirus serotypes and antibodies that bind to human rotavirus and modifications thereto which enhance the immunogenicity of the rotavirus protein for vaccine development with respect to the generation of a neutralizing immune response. Further disclosed are methods of using the antibodies for treating a rotavirus-mediated disease in a subject.

### CROSS REFERENCE

This application claims benefit and is a 371 application of PCT Application No. PCT/US2016/041613, filed Jul. 8, 2016, which claims benefit of U.S. Provisional Patent Application No. 62/189,901, filed Jul. 8, 2015, which applications are incorporated herein by reference in their entirety.

### FEDERALLY SPONSORED RESEARCH AND DEVELOPMENT

This invention was made with Government support under contract AI021362 awarded by the National Institutes of Health. The Government has certain rights in the invention.

### BACKGROUND

Human rotaviruses (RV) are the leading cause of severe and fatal diarrhea in infants and children less than 5 years of age, and account for between 200,00 and 400,000 global deaths per year. There are two safe and effective, widely licensed second-generation RV vaccines, RotaTeq (Merck) and Rotarix (Glaxo Smith Kline), however the serologic basis for the efficacy of both vaccines is unknown. Furthermore, both vaccines demonstrate reduced (33-70%) efficacy in developing countries where the burden of RV disease is greatest, compared to high efficacy in developed countries (>90%).

RVs are non-enveloped dsRNA viruses characterized by a triple-layered protein capsid comprised of two surface proteins (VP4, VP7), a major inner protein (VP6), and an 11-segmented dsRNA genome encoding at least 12 gene products, including 6 nonstructural proteins. RVs infect enterocytes of the small-intestinal villi and replicate exclusively in the cytoplasm. Virus infectivity is increased by proteolytic cleavage of the trimeric spike protein, VP4, to yield its stalk (VP5*) and globular head (VP8*) subunits. These cleavage products remain non-covalently associated on the mature virion surface. VP8* mediates attachment to host cell glycoconjugates, while VP5* facilitates membrane penetration. VP7 is the main glycoprotein constituent of the outer capsid of the mature virion.

RVs have vast serotypic diversity due to independent segregation of VP4 and VP7 by gene reassortment and antigenic differences in these proteins that provide the basis for strain classification into G (VP7) and P (VP4) serotypes. A total of 15 G serotypes and 22 P genotypes have been described. Although the majority of prevailing human RV worldwide have G1, G2, G3, G4 and G9 as the G serotype and P[4], P[6] and P[8] as the P genotype, at least 10 G and 10 P types have been reported on human RVs. In recent years an increasing number of human RVs with unusual G or P types and rare combinations of G and P types have been reported worldwide.

Protective immunity to RV is primarily mediated through neutralizing antibodies (Abs) that target epitopes in VP4 and VP7. Genetic and passive Ab transfer studies have shown that VP4 and VP7 are the only targets of in vitro neutralization and that feeding neutralizing Abs to either protein protects mice from homotypic and/or heterotypic challenge. The atomic and related antigenic structures of VP4 and VP7 have been elucidated; both proteins contain conformationally-dependent regions that stimulate homotypic (serotype specific) as well as heterotypic (serotype cross-reactive) immunity. However, the characterization of heterotypic versus homotypic interactions of VP4 and VP7 neutralizing human Abs with the virion have not been fully studied.

Specifically, in humans it is not known whether broadly protective immunity is mediated by individual anti-VP4 and -VP7 Ig molecules with heterotypic cross-reactivity or by an array of individual Igs each with restricted specificities against serotypically distinct RV antigens. Epidemiologic studies and clinical trials worldwide support the former hypothesis as they have demonstrated that a single RV infection or vaccination with the monovalent Rotarix or Rotavac vaccine (G1,P[8] or G911] respectively), is sufficient to induce broad heterotypic protective immunity. This conclusion is reinforced by the comparable efficacy of the monovalent Rotarix and the pentavalent Rotateq vaccine, the latter of which contains five live reassortant RVs each expressing serotypically distinct RV antigens. Only one molecular study in humans analyzed RV-specific Ab clones generated from a bone marrow-derived phage display library. Among the twelve clones analyzed three neutralizing human mAbs were identified; two VP4 mAbs isolated had heterotypic specificities whereas the single VP7 mAb had only homotypic specificity.

A significant challenge for vaccine development is defining conserved epitopes that are capable of eliciting cross-reactive protective antibodies in this highly diverse virus. Treatment of rotavirus and the development of vaccines that broadly protect against highly diverse rotavirus serotypes are of interest in the field, particularly due to the fact that lowered protective immunity to current, licensed vaccine formulations is low in regions of the world with the highest proportion or morbidity and mortality attributed to rotavirus. The present invention addresses this issue.

### SUMMARY

Human recombinant, neutralizing monoclonal antibodies (mAbs) specific to rotavirus protein epitopes are provided. The provided antibodies were generated by cloning natively paired heavy (IgH) and light (IgL) chain antibody (Ab) genes derived from effector B cells in the small intestinal mucosa of RV-experienced adults. In some embodiments, the antibodies have heterotypic (serotype cross-reactive) neutralizing capacity against two, three or more RV strains. In some embodiments the antibodies have homotypic (serotype specific) neutralizing activity. In some embodiments the antibody is specific for a VP7 epitope. In some embodiments the antibody is specific for a VP4 epitope, including the VP5* cleavage product of VP4. Exemplary antibody sequences are provided herein.

These mAbs are useful in defining epitopes that stimulate homotypic versus heterotypic protective immunity in humans, and in the rational design of more effective RV vaccines, for example vaccines that include epitopes that stimulate heterotypic immunity against serotypically distinct RV strains circulating worldwide. These antibodies are also therapeutically useful.

The antibodies provided herein include VP4 specific mAbs, including without limitation antibodies specific for the VP5* cleavage product, that neutralyze RVs with diverse serotypes, including G×P6, G×P8, G×P4 and G×P3. The native antibodies are typically of an IgA isotype; in some embodiments the antibody is provided as an antibody of other than IgA isotype, e.g. IgG1, IgG2a, IgG2b, IgG3, IgG4; as a single chain antibody; in combination with an engineered Fc region, and the like. The antibody may be labeled with a detectable label, immobilized on a solid phase and/or conjugated with a heterologous compound. The antibody or a cocktail of antibodies may be provided as a pharmaceutical formulation

Embodiments of the invention include isolated antibodies and derivatives and fragments thereof, pharmaceutical formulations comprising one or more of the human anti-rotavirus monoclonal antibodies; and cell lines that produce these monoclonal antibodies. Also provided are CDR amino acid sequences that confer the binding specificity of these monoclonal antibodies. These sequences and the cognate epitopes to which the monoclonal antibodies of the invention bind can be used to identify other antibodies that specifically bind and neutralize rotavirus; including without limitation epitopes of VP5*, and immunotherapeutic methods for prevention of disease associated with RV. An advantage of the monoclonal antibodies of the invention derives from the fact that they are encoded by a human polynucleotide sequence.

Thus, in vivo use of the monoclonal antibodies of the invention for immunotherapy greatly reduces the problems of significant host immune response to the passively administered antibodies. Therapies of interest include combination therapies with anti-rotavirus therapeutics such as rehydration therapy, and the like.

The human anti-rotavirus antibody may have a heavy chain variable region comprising the amino acid sequence of CDR1 and/or CDR2 and/or CDR3 of the provided monoclonal antibodies as provided herein; and/or a light chain variable region comprising the amino acid sequence of CDR1 and/or CDR2 and/or CDR3 of the provided human monoclonal human antibodies as provided herein. In other embodiments, the antibody comprises an amino acid sequence variant of one or more of the CDRs of the provided human antibodies, which variant comprises one or more amino acid insertion(s) within or adjacent to a CDR residue and/or deletion(s) within or adjacent to a CDR residue and/or substitution(s) of CDR residue(s) (with substitution(s) being the preferred type of amino acid alteration for generating such variants). Such variants will normally having a high binding affinity for rotavirus VP4 or VP7.

Diagnostic and therapeutic uses for the antibody are contemplated. In one diagnostic application, the invention provides a method for determining the presence of a specific serotype of human rotavirus virus exposing a sample suspected of containing the rotavirus virus to the anti-rotavirus antibody and determining binding of the antibody to the sample. While human VP6-specific mAbs have been identified in the past that would serve to identify the presence of rotavirus, VP4- and VP7-specific mAbs that react with specific serotypes would allow for diagnosis of the specific infecting human strain.

The invention further provides: isolated nucleic acid encoding the antibodies and variants; a vector comprising that nucleic acid, optionally operably linked to control sequences recognized by a host cell transformed with the vector; a host cell comprising that vector; a process for producing the antibody comprising culturing the host cell so that the nucleic acid is expressed and, optionally, recovering the antibody from the host cell culture (e.g. from the host cell culture medium). The invention also provides a composition comprising one or more of the human anti-rotavirus antibodies and a pharmaceutically acceptable carrier or diluent. This composition for therapeutic use is sterile and may be lyophilized, e.g. being provided as a pre-pack in a unit dose with diluent and delivery device, e.g. inhaler, syringe, etc.

A basis for heterotypic neutralizing reactivity to RV in humans at the individual immunoglobulin (Ig) molecule level is identified. In some embodiments a method of defining such activity is provided, comprising the steps of sorting single cells of intestinal RV-specific IgA⁺ antibody secreting cells, by contacting the cells with triple-layered RV particles conjugated to a detectable label, e.g. a fluorochrome suitable for sorting by flow cytometry. The immunoglobulin coding polynucleotides from such sorted cells are sequenced with an identifying barcode. The antibodies thus identified by sequences are tested for activity in RV neutralization in vitro against two or more different RV serotypes, where antibodies that neutralize multiple serotypes are defined as heterotypic antibodies. The methods are useful in providing detailed analysis of thre ability of an immunogen, e.g. a vaccine, to elicit a protective heterotypic response. Humans can circumvent the serotypic diversity of naturally circulating RV strains by expressing individual VP4 epitope-specific Ig molecules that mediate heterotypic neutralization. Characterization of the structural targets of these mAbs, and determination of the extent to which they arise following primary RV infection of children provide the basis for designing more effective RV vaccines.

Antigenic compositions are provided, which comprise all or a portion of a rotavirus protein in which specific highly immunodominant residues are masked or deleted, so as to generate an immune response to residues that are less immunodominant, but which are essential for virus function and therefore are less likely to be altered in virus escape mutation and selection. Alternatively antigenic compositions providing epitopes for heterotypic neutralizing antibodies are provided, which can be formulated alone or in combination with conventional vaccines. Antigens may comprise, without limitation, VP5* proteins, alone or in combination with an adjuvant. These antigens find use in screening assays, generation of monoclonal antibodies, and in vaccines. Such formulations may comprise, without limitation, live attenuated formulation containing known heterotypic neutralizing epitopes (and excluding known homotypic neutralizing epitopes); and/or epitope immunogens with known heterotypic neutralizing epitopes or overlapping neutralizing epitopes. These novel vaccines/immunogens could be used in combination with current formulations, for example in a prime boost strategy to enhance immunity in children and infants who do not respond to the current, licensed vaccines or formulations alone. The formulations of the invention may find particular benefit in providing improved protective immunity in regions of the world with the highest RV disease burden and lowest vaccine efficacy observed in several clinical trials of the current licensed RV vaccines.

In some embodiments of the invention, a modified rotavirus VP4, including a VP5* fragment, or VP7 polypeptide is provided, which provides for enhanced heterotypic immune responsiveness In other embodiments, a polynucleotide encoding such a modified rotavirus polypeptide is provided. The polypeptide and/or the nucleic acid can be used in the formulation of a vaccine, e.g. a virus-like particle, a recombinant protein vaccine which can be formulated with an adjuvant, a vector vaccine, and the like. In some embodiments, a vaccine formulation comprising a polypeptide or a polynucleotide of the invention is provided.

Other aspects and features will be readily apparent to the ordinarily skilled artisan upon reading the present disclosure.

### BRIEF DESCRIPTION OF THE DRAWINGS

The invention is best understood from the following detailed description of exemplary embodiments when read in conjunction with the accompanying drawings. It is emphasized that, according to common practice, the various features of the drawings are not necessarily to-scale. On the contrary, the dimensions of the various features are arbitrarily expanded or reduced for clarity. Included in the drawings are the following figures:

FIG. 1A-1E. Identification of RV-specific antibody secreting B cells by flow cytometry using triple layered particles (TLP)-Cy5. (FIG. 1A) RV TLPs (CDC-9 strain, G1, P[8]) were labeled with Cy5 and the structural integrity of the TLPs-Cy5 determined by electron microscopy TLP-Cy5 specifically stained G1- and P8-specific murine hybridomas but did not stain G3- and P3-specific hybridomas by FACS. (FIG. 1B) TLP-Cy5 binding to G1- and P8-specific hybridomas was reduced by blocking with unlabeled TLPs. TLP-Cy5 bound VP6-specific hybridoma; Treatment of TLP-Cy5 with 5 mM EDTA increased the proportion of VP6-specific hybridoma cells that stained positive by FACS compared to untreated TLP-Cy5. (FIG. 1C) Shown are represented histogram overlays and the mean fluorescence intensity+SD from two independent experiments. At least 100,000 events were acquired per sample. (FIG. 1D) Human intestinal ASCs were identified by FACS based on single, live, CD3/14/16⁻ CD20^{lo/-} CD27^{hi} CD38^{hi} surface phenotype. TLP-Cy5-binding B cells were gated based on unstained cells. Blocking with unlabeled TLPs reduced TLP-Cy5-specific staining on intestinal ASCs. Shown are FACS plots from a representative donor and the mean frequency of intestinal TLP⁺ ASCs±SD from repeated experiments on two donors. At least 200,000 events were acquired per sample; and (FIG. 1E) the mean frequency of intestinal TLP. ASCs+SD from repeated experiments on two donors. At least 200,000 events were acquired per sample. P values were obtained using one-way ANOVA. *, P<0.05; **, P<0.01; ****, P<0.0001.

FIG. 2A-2C. Identification and frequency of TLP-binding human intestinal ASCs at steady-state in adults donors. (FIG. 2A) Gating strategy used to identify CDC-9 TLP-binding intestinal ASCs derived from proximal jejunum tissue resections of adult donors. Live, single cells were gated based on CD3/14/16⁻ CD20^{hi/-} CD27^{hi} CD38^{hi} IgA⁺ surface expression. Shown are contour plots from a representative donor. At least 200,000 events were acquired per sample. (FIG. 2B) The frequency of IgA+ and IgA− ASCs as a proportion of total intestinal ASCs (left) and the frequency of TLP-binding IgA+ and IgA− ASCs as a proportion of total IgA+ and IgA− ASCs (right), as determined by FACS are shown. (FIG. 2C) The frequency of IgA+ ASCs as a proportion of total intestinal B cells (left) and the frequency of DLP-binding IgA+ ASCs as a proportion of total IgA+ ASCs (right) as determined by ELISPOT. Symbols represent the frequencies of individual donors as shown in the legend to the right. Red lines represent the median frequencies from five donors. P values were obtained using the unpaired t test. * P<0.05.

FIG. 3A-3E. Phylogenetic tree of the RV TLP-reactive IgA₊ ASC intestinal Ab repertoire. Combined heavy and light chain dendrograms of the Ab repertoires of TLP-binding intestinal IgA₊ ASCs from five donors. The subject ID and the total number of paired Ab sequences used to generate each phylogenetic tree are shown in the center. Each peripheral node depicts a sequenced VH and VL region derived from a single cell. Colors indicate VH gene families as indicated in the legend to the right and red lines indicate clonal families. Ig V gene sequences that were selected for cloning and expression of recombinant mAbs are numbered. Stars denote Abs that bound RV proteins, circled red stars denote neutralizing Abs, and squares denote Abs that did not bind RV.

FIG. 4A-4E. Molecular characteristics of paired IgH and IgL immunoglobulin genes expressed by individual TLP-reactive intestinal IgA₊ ASCs. (FIG. 4A) Heatmap representation of VH-VL combinations that occurred in more than one donor among IgA+ ASC Ab sequences. Colors indicate the sequence-normalized number of Abs per combination as shown in the scale below the heatmap. (FIG. 4B) The frequency of replacement (black) and silent (white) mutations in FWRs and CDRs of the 821 IgA+ ASC gene sequences analyzed. (FIG. 4C) The absolute number of somatic mutations in VH (n=821), VK (n=413), VL (n=407) genes of the IgA+ ASC gene sequences and (FIG. 4D) Lengths of CDR3 regions encoded by IgA VH (n=821), VK (n=413) and VL (n=407) genes. Red circles denote sequences from mAbs that bound RV proteins, black triangles denote neutralizing Abs, and gray circles denote all other Ab sequences. (FIG. 4E) Frequency of positively charged amino acids in CDRH3 regions of the 821 IgA gene sequences. Black lines represent the median values from all sequences. The absolute number of sequences analyzed from all donors is indicated over each graph. P values were obtained using one way ANOVA. ****, P<0.0001.

FIG. 5A-5C. Recombinant human mAbs can mediate heterotypic as well as homotypic protection from RV-induced diarrheal disease. In vivo protection of RV induced diarrheal disease by mAb #27, mAb #57 and mAb #41. Shown is the percent of diarrheal disease following inoculation of 5 day old 129sv suckling mice (6-8 mice per group) challenged with 10⁶ PFU of indicated RV strains pre-incubated for 1 hr with 5 ug/ml of indicated human (FIG. 5A, 5B) anti-VP7, (FIG. 5C) anti-VP4 or with media control. The percentage of pups with diarrhea was assessed for 4 days post challenge.

### DETAILED DESCRIPTION OF THE EMBODIMENTS

It is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the polypeptide” includes reference to one or more polypeptides and equivalents thereof known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

As used herein, the terms “neutralizes rotavirus,” “inhibits rotavirus,” and “blocks rotavirus” are used interchangeably to refer to the ability of an antibody of the invention to prevent rotavirus from infecting a given cell.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein a “heterotypic” antibody is capable of neutralizing two, three, four or more different rotavirus serotypes. A “homotypic” antibody neutralizes specifically a single serotype, particularly the serotype used as an immunogen.

The term “rotavirus protein” includes without limitation the proteins, particularly VP4, VP7 and fragments thereof, of known serotypes that infect humans, e.g. as described in Hemming and Vesikari (2013) Infect Genet Evol. October; 19:51-8; Lahon and Chitambar (2011) Asian Pac J Trop Med. November; 4(11):846-9; Arora et al. (2011) Asian Pac J Trop Med. July; 4(7):541-6; Aung et al. (2009) J Med Virol. 2009 November; 81(11):1968-74; Yoder et al. (2009) J Virol. 2009 November; 83(21):11372-7, each herein specifically incorporated by reference.

VP5* epitopes are shown herein to be associated with heterotypic antibody responses. Proteolytic cleavage of the VP4 outer capsid spike protein into VP8* and VP5* proteins is required for rotavirus infectivity and for rotavirus-induced membrane permeability. The cleavage site may be at about amino acid 247-248 of VP4, thus the VP5* fragment may comprise from about residue 247 to about residue 775. A recombinant VP5* fragment has a trimeric, folded-back structure. VP5* forms the spike body and foot and is thought to mediate membrane penetration. The head and body domains form an asymmetrical dyad that protrudes from the VP7 shell.

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

“Polypeptide” and “protein” as used interchangeably herein, can encompass peptides and oligopeptides. Where “polypeptide” is recited herein to refer to an amino acid sequence of a naturally-occurring protein molecule, “polypeptide” and like terms are not necessarily limited to the amino acid sequence to the complete, native amino acid sequence associated with the recited protein molecule, but instead can encompass biologically active variants or fragments, including polypeptides having substantial sequence similarity or sequence identify relative to the amino acid sequences provided herein. In general, fragments or variants retain a biological activity of the parent polypeptide from which their sequence is derived.

As used herein, “polypeptide” refers to an amino acid sequence of a recombinant or non-recombinant polypeptide having an amino acid sequence of i) a native polypeptide, ii) a biologically active fragment of an polypeptide, or iii) a biologically active variant of an polypeptide. Polypeptides suitable for use can be obtained from any species, e.g., mammalian or non-mammalian (e.g., reptiles, amphibians, avian (e.g., chicken)), particularly mammalian, including human, rodent (e.g., murine or rat), bovine, ovine, porcine, murine, or equine, particularly rat or human, from any source whether natural, synthetic, semi-synthetic or recombinant. In general, polypeptides comprising a sequence of a human polypeptide are of particular interest.

The term “derived from” indicates molecule that is obtained directly from the indicated source (e.g., when a protein directly purified from a cell, the protein is “derived from” the cell) or information is obtained from the source, e.g. nucleotide or amino acid sequence, from which the molecule can be synthesized from materials other than the source of information.

The term “isolated” indicates that the recited material (e.g, polypeptide, nucleic acid, etc.) is substantially separated from, or enriched relative to, other materials with which it occurs in nature (e.g., in a cell). A material (e.g., polypeptide, nucleic acid, etc.) that is isolated constitutes at least about 0.1%, at least about 0.5%, at least about 1% or at least about 5% by weight of the total material of the same type (e.g., total protein, total nucleic acid) in a given sample.

The terms “subject” and “patient” are used interchangeably herein to mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest. As will be evidence from the context in which the term is used, subject and patient refer to a subject or patient susceptible to infection by a Flaviviridae virus, particularly rotavirus.

“Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The term “unit dosage form,” as used herein, refers to physically discrete units suitable as unitary dosages for human and animal subjects, each unit containing a predetermined quantity of compounds calculated in an amount sufficient to produce the desired effect in association with a pharmaceutically acceptable diluent, carrier or vehicle. The specifications for the novel unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and is usually free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like.

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Unless specifically indicated to the contrary, the term “conjugate” as described and claimed herein is defined as a heterogeneous molecule formed by the covalent attachment of one or more polypeptide fragment(s) to one or more polymer molecule(s), wherein the heterogeneous molecule is water soluble, i.e. soluble in physiological fluids such as blood, and wherein the heterogeneous molecule is free of any structured aggregate. A conjugate of interest is PEG. In the context of the foregoing definition, the term “structured aggregate” refers to (1) any aggregate of molecules in aqueous solution having a spheroid or spheroid shell structure, such that the heterogeneous molecule is not in a micelle or other emulsion structure, and is not anchored to a lipid bilayer, vesicle or liposome; and (2) any aggregate of molecules in solid or insolubilized form, such as a chromatography bead matrix, that does not release the heterogeneous molecule into solution upon contact with an aqueous phase. Accordingly, the term “conjugate” as defined herein encompasses the aforementioned heterogeneous molecule in a precipitate, sediment, bioerodible matrix or other solid capable of releasing the heterogeneous molecule into aqueous solution upon hydration of the solid.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

By the term “vaccine” as used herein, is meant a composition; a formulation comprising a modified polypeptide of the invention; a virus or virus-like particle comprising a modified polypeptide of the invention complex; or a DNA encoding a modified polypeptide of the invention complex, which, when administered to a subject, induces cellular or humoral immune responses as described herein.

Some embodiments of the invention provide a method of stimulating an immune response in a mammal, which can be a human or a preclinical model for human disease, e.g. mouse, ape, monkey etc. “Stimulating an immune response” includes, but is not limited to, inducing a therapeutic or prophylactic effect that is mediated by the immune system of the mammal. More specifically, stimulating an immune response in the context of the invention refers to eliciting cellular or humoral immune responses, thereby inducing downstream effects such as production of antibodies, antibody heavy chain class switching, maturation of APCs, and stimulation of cytolytic T cells, T helper cells and both T and B memory cells.

As appreciated by skilled artisans, vaccine compositions are suitably formulated to be compatible with the intended route of administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of the composition can be adjusted with acids or bases, such as hydrochloric acid or sodium hydroxide. Systemic administration of the composition is also suitably accomplished by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

Vaccine compositions may include an aqueous medium, pharmaceutically acceptable inert excipient such as lactose, starch, calcium carbonate, and sodium citrate. Vaccine compositions may also include an adjuvant, for example Freud's adjuvant. Vaccines may be administered alone or in combination with a physiologically acceptable vehicle that is suitable for administration to humans. Vaccines may be delivered orally, parenterally, intramuscularly, intranasally or intravenously. Oral delivery may encompass, for example, adding the compositions to the feed or drink of the mammals. Factors bearing on the vaccine dosage include, for example, the weight and age of the mammal. Compositions for parenteral or intravenous delivery may also include emulsifying or suspending agents or diluents to control the delivery and dose amount of the vaccine.

Modified polypeptides of the invention and polynucleotides that encode such modified polypeptides can be used in various rotavirus vaccine formulations known in the art, as a substitution for the wild-type rotavirus sequence. Polypeptides can be fragmented to generate a peptide vaccine, e.g. administered with poly-L-arginine, can be formulated as a vaccine. Polynucleotides encoding modified polypeptides can be administered in virus form, e.g. modified rotavirus, plasmid form, in a virus genome, including adenovirus, alphaviruses, canary pox, ovine atadenovirus and semliki-like viral particles. Advances in molecular virology have enabled the manipulation of viruses for delivery of foreign genetic material to mammalian cells. Their highly evolved mechanisms for cell entry and gene expression within the host cell remain intact and viral vectors can be rendered non-pathogenic and non-replicative by deletions at specific locus.

Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VH) domain. The two domains conventionally associate to form a specific binding region, although a variety of non-natural configurations of antibodies are known and used in the art.

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or enzymatic activity. A functional antibody or other binding molecule may neutralize a virus particle. The capability of an antibody or other binding molecule to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. In some embodiments the antibody is other than a full length IgA antibody. In one aspect, the antibody is of largely human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include at least one CDR sequence from the variable regions provided herein, usually at least 2 CDR sequences, and more usually 3 CDR sequences. exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlanet al. “Identification of specificity-determining residues in antibodies.” Faseb J. 1995; 9:133-139., each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called K and A, based on the amino acid sequences of their constant domains.

A “functional Fc region” possesses an “effector function” of a native-sequence Fc region. Exemplary effector functions include C1q binding; CDC; Fc-receptor binding; ADCC; ADCP; down-regulation of cell-surface receptors (e.g., B-cell receptor), etc. Such effector functions generally require the Fc region to be interact with a receptor, e.g. the FcγRI; FcγRIIA; FcγRIIB1; FcγRIIB2; FcγRIIIA; FcγRIIIB receptors, and the law affinity FcRn receptor; and can be assessed using various assays as disclosed, for example, in definitions herein. A “dead” Fc is one that has been mutagenized to retain activity with respect to, for example, prolonging serum half-life, but which does not activate a high affinity Fc receptor.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

“Fv” is the minimum antibody fragment, which contains a complete antigen-recognition and antigen-binding site. The CD3 binding antibodies of the invention comprise a dimer of one heavy chain and one light chain variable domain in tight, non-covalent association; however additional antibodies, e.g. for use in a multi-specific configuration, may comprise a VH in the absence of a VL sequence. Even a single variable domain (or half of an Fv comprising only three hypervariable regions specific for an antigen) has the ability to recognize and bind antigen, although the affinity may be lower than that of two domain binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The term “single chain antibody” as used herein means a single polypeptide chain containing one or more antigen binding domains that bind an epitope of an antigen, where such domains are derived from or have sequence identity with the variable region of an antibody heavy or light chain. Parts of such variable region may be encoded by V_{H} or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments. V-, D- and J-gene segments may be derived from humans and various animals including birds, fish, sharks, mammals, rodents, non-human primates, camels, lamas, rabbits and the like.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

Other definitions of terms appear throughout the specification.

### Antibody Compositions

Compositions and methods are provided relating to human anti-rotavirus monoclonal antibodies. The antibodies of the invention bind to and neutralize rotavirus virus across multiple genotypes. Embodiments of the invention include isolated antibodies and derivatives and fragments thereof, pharmaceutical formulations comprising one or more of the human anti-rotavirus monoclonal antibodies; cell lines that produce these monoclonal antibodies.

In one aspect, the present invention is directed to combinatorially derived human monoclonal antibodies which are specifically reactive with and neutralize rotavirus, and cell lines which produce such antibodies. Variable regions of exemplary antibodies are provided, e.g. SEQ ID NO:19-36 provide protein sequences of antibodies, which may be paired, as intact variables regions or as a set of CDR sequences derived therefrom, as SEQ ID NO:19 and 20; SEQ ID NO:21 and 22; SEQ ID NO:23 and 24; SEQ ID NO:25 and 26; SEQ ID NO:27 and 28; SEQ ID NO:29 and 30; SEQ ID NO:31 and 32; SEQ ID NO:33 and 34; and SEQ ID NO:35 and 36. Combinations of particular interest include those antibodies shown to have heterotypic neutralization activity, i.e. mAb ID nos. 2, 30, 41, 47, 49 and 57, which correspond to the combinations as intact variables regions or as a set of CDR sequences derived therefrom SEQ ID NO:19 and 20; SEQ ID NO:23 and 24; SEQ ID NO:27 and 28; SEQ ID NO:31 and 32; SEQ ID NO:33 and 34; and SEQ ID NO:35 and 36.

Antibodies of interest include these provided combinations, as well as fusions of the variable regions to appropriate constant regions or fragments of constant regions, e.g. to generate F(ab)′ antibodies. Variable regions of interest include at least one CDR sequence, where a CDR may be 3, 4, 5, 6, 7, 8, 9, 10, 11, 12 or more amino acids. Alternatively, antibodies of interest include a pair of variable regions. In other embodiments, one antibody chain can comprise the set of CDR sequences from a heavy chain. Such an antibody chain may be combined with an antibody chain comprising the set of CDR sequences from a light chain. Certain antibodies of the invention bind to rotavirus VP4, including the VP5* fragment, or VP7 proteins of different rotavirus serotypes.

One or more residues of a CDR may be altered to modify binding to achieve a more favored on-rate of binding, a more favored off-rate of binding, or both, such that an optimized binding constant is achieved. Affinity maturation techniques are well known in the art and can be used to alter the CDR region(s), followed by screening of the resultant binding molecules for the desired change in binding. In addition to, or instead of, modifications within the CDRs, modifications can also be made within one or more of the framework regions, FRI, FR2, FR3 and FR4, of the heavy and/or the light chain variable regions of a human antibody, so long as these modifications do not eliminate the binding affinity of the human antibody.

In general, the framework regions of human antibodies are usually substantially identical, and more usually, identical to the framework regions of the human germline sequences from which they were derived. Of course, many of the amino acids in the framework region make little or no direct contribution to the specificity or affinity of an antibody. Thus, many individual conservative substitutions of framework residues can be tolerated without appreciable change of the specificity or affinity of the resulting human immunoglobulin. Thus, in one embodiment the variable framework region of the human antibody shares at least 85% sequence identity to a human germline variable framework region sequence or consensus of such sequences. In another embodiment, the variable framework region of the human antibody shares at least 90%, 95%, 96%, 97%, 98% or 99% sequence identity to a human germline variable framework region sequence or consensus of such sequences. In addition to simply binding a linear epitope of an rotavirus protein, a monoclonal antibody may be selected for its retention of other functional properties of antibodies of the invention, such as binding to multiple serotypes of rotavirus and/or binding with an ultra-high affinity such as, for example, a K_{D} of 10^{*9} M or lower.

In some embodiments a polypeptide of interest has a contiguous sequence of at least about 10 amino acids as set forth in any one of sequences provided herein, at least about 15 amino acids, at least about 20 amino acids, at least about 25 amino acids, at least about 30 amino acids, up to the complete provided variable region. Polypeptides of interest also include variable regions sequences that differ by up to one, up to two, up to 3, up to 4, up to 5, up to 6 or more amino acids as compared to the amino acids sequence set forth in any one of sequences provided herein. In other embodiments a polypeptide of interest is at least about 80%, at least about 85%, at least about 90%, at least about 95%, at least about 99% identical to the amino acid sequence set forth in any one of sequences provided herein.

The isolation of cells producing monoclonal antibodies of the invention can be accomplished using routine screening techniques, which permit determination of the elementary reaction pattern of the monoclonal antibody of interest. Thus, if a human monoclonal antibody being tested binds to the cognate epitope of one of the provided antibodies, i.e. cross-blocks, and neutralizes rotavirus, then the human monoclonal antibody being tested and the human monoclonal antibody exemplified herein are equivalent.

It is also possible to determine, without undue experimentation, if a human monoclonal antibody has the same specificity as a human monoclonal antibody of the invention by ascertaining whether the former prevents the latter from binding to or neutralizing rotavirus, including without limitation an ability to neutralize an rotavirus virus of multiple serotypes. If the human monoclonal antibody being tested competes with the human monoclonal antibody of the invention, as shown by a decrease in binding by the human monoclonal antibody of the invention, then the two monoclonal antibodies bind to the same, or a closely related, epitope. Still another way to determine whether a human monoclonal antibody has the specificity of a human monoclonal antibody of the invention is to pre-incubate the human monoclonal antibody of the invention with rotavirus with which it is normally reactive, and then add the human monoclonal antibody being tested to determine if the human monoclonal antibody being tested is inhibited in its ability to bind rotavirus. If the human monoclonal antibody being tested is inhibited then, in all likelihood, it has the same, or functionally equivalent, epitopic specificity as the monoclonal antibody of the invention. Screening of human monoclonal antibodies of the invention can be also carried out utilizing rotavirus and determining whether the monoclonal antibody neutralizes rotavirus.

In addition to Fabs, smaller antibody fragments and epitope-binding peptides having binding specificity for at least one epitope of rotavirus are also contemplated by the present invention and can also be used to neutralize the virus. For example, single chain antibodies can be constructed according to the method of U.S. Pat. No. 4,946,778 to Ladner et al, which is incorporated herein by reference in its entirety. Single chain antibodies comprise the variable regions of the light and heavy chains joined by a flexible linker moiety. Yet smaller is the antibody fragment known as the single domain antibody, which comprises an isolate VH single domain. Techniques for obtaining a single domain antibody with at least some of the binding specificity of the intact antibody from which they are derived are known in the art. For instance, Ward, et al. in “Binding Activities of a Repertoire of Single Immunoglobulin Variable Domains Secreted from Escherichia coli,” Nature 341: 644-646, disclose a method for screening to obtain an antibody heavy chain variable region (H single domain antibody) with sufficient affinity for its target epitope to bind thereto in isolate form.

### Methods of Use

The invention includes methods of treating an rotavirus-mediated disease in a subject by administering to the subject an isolated human monoclonal antibody or antigen binding portion thereof as described herein (i.e., that specifically binds to rotavirus), or a cocktail of such antibodies, in an amount effective to inhibit rotavirus disease, e.g., rotavirus-mediated symptoms or morbidity. Such diseases may include various conditions associated with rotavirus infection such as severe dehydrating diarrhea. Treatment may include the use of the monoclonal antibodies of the invention as a single agent, or as an agent in combination with rehydration therapy, drugs, additional antibodies, vaccines, and the like.

Subjects suspected of having an rotavirus infection can be screened prior to therapy. Further, subjects receiving therapy may be tested in order to assay the activity and efficacy of the treatment. Significant improvements in one or more parameters is indicative of efficacy. It is well within the skill of the ordinary healthcare worker (e.g., clinician) to adjust dosage regimen and dose amounts to provide for optimal benefit to the patient according to a variety of factors (e.g., patient-dependent factors such as the severity of the disease and the like, the compound administered, and the like). For example, rotavirus infection in an individual can be detected and/or monitored by the presence of rotavirus RNA in blood, and/or having anti-rotavirus antibody in their serum.

Subjects for whom the therapy disclosed herein is of interest include subject who are “difficult to treat” subjects due to the nature of the rotavirus infection or the nature of the individual, e.g. extreme youth or age, immunosuppression, etc.

Human monoclonal antibodies or portions thereof (and compositions comprising the antibodies or portions thereof) of the invention can be administered in a variety of suitable fashions, e.g., intravenously (IV), subcutaneously (SC), or, intramuscularly (IM) to the subject. The antibody or antigen-binding portion thereof can be administered alone or in combination with another therapeutic agent, e.g., a second human monoclonal antibody or antigen binding portion thereof. In one example, the second human monoclonal antibody or antigen binding portion thereof specifically binds to a second rotavirus isolate that differs from the isolate bound to the first antibody. In another example, the antibody is administered together with another agent, for example, an antiviral agent. Antiviral agents includes pegylated interferon α, ribivarin, etc. In another example, the antibody is administered together with a polyclonal gamma-globulin (e.g., human gammaglobulin). In another example, the antibody is administered before, after, or contemporaneously with a rotavirus vaccine.

The human monoclonal antibodies of the invention can be used in vitro and in vivo to detect or monitor the course of rotavirus disease. Thus, for example, by measuring the increase or decrease in the number of cells infected with rotavirus or changes in the concentration of rotavirus present in the body or in various body fluids, it would be possible to determine whether the presence of disease, the course of disease, and/or whether a particular therapeutic regimen aimed at ameliorating the rotavirus disease is effective.

The monoclonal antibodies of the invention may be used in vitro in immunoassays in which they can be utilized in liquid phase or bound to a solid phase carrier. In addition, the monoclonal antibodies in these immunoassays can be detectably labeled in various ways. Examples of types of immunoassays which can utilize monoclonal antibodies of the invention are competitive and non-competitive immunoassays in either a direct or indirect format. Examples of such immunoassays are the radioimmunoassay (RIA) and the sandwich (immunometric) assay. Detection of the antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The monoclonal antibodies of the invention can be bound to many different carriers and used to detect the presence of rotavirus. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, and bio-luminescent compounds. Those of ordinary skill in the art will know of other suitable labels for binding to the monoclonal antibodies of the invention, or will be able to ascertain such, using routine experimentation. Furthermore, the binding of these labels to the monoclonal antibodies of the invention can be done using standard techniques common to those of ordinary skill in the art.

For purposes of the invention, human rotavirus may be detected by the monoclonal antibodies of the invention when present in biological fluids and tissues. Any sample containing a detectable amount of rotavirus can be used. A sample can be a liquid such as urine, saliva, cerebrospinal fluid, blood, serum and the like, or a solid or semi-solid such as tissues, feces, and the like, or, alternatively, a solid tissue such as those commonly used in histological diagnosis.

Another labeling technique which may result in greater sensitivity consists of coupling the antibodies to low molecular weight haptens. These haptens can then be specifically detected by means of a second reaction. For example, it is common to use haptens such as biotin, which reacts with avidin, or dinitrophenol, pyridoxal, or fluorescein, which can react with specific anti-hapten antibodies.

As a matter of convenience, the antibody of the present invention can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### Polynucleotides

The invention also provides isolated nucleic acids encoding the human anti-rotavirus antibodies, vectors and host cells comprising the nucleic acid, and recombinant techniques for the production of the antibody. Exemplary polynucleotides encode the heavy or light chain variable region sequences set forth herein, e.g. SEQ ID NO:1-18.

Nucleic acids of interest may be at least about 80% identical to a sequence that encodes SEQ ID NO:1-18, at least about 85%, at least about 90%, at least about 95%, at least about 99%, or identical. In some embodiments a contiguous nucleotide sequence is at least about 20 nt, at least about 25 nt, at least about 50 nt, at least about 75 nt, at least about 100 nt, and up to the complete coding sequence may be used. Such contiguous sequences may encode a CDR sequence, or may encode a complete variable region. As is known in the art, a variable region sequence may be fused to any appropriate constant region sequence.

For recombinant production of the antibody, the nucleic acid encoding it is inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

The anti-rotavirus antibody of this invention may be produced recombinantly not only directly, but also as a fusion polypeptide with a heterologous or homologous polypeptide, which include a signal sequence or other polypeptide having a specific cleavage site at the N-terminus of the mature protein or polypeptide, an immunoglobulin constant region sequence, and the like. A heterologous signal sequence selected preferably may be one that is recognized and processed (i.e., cleaved by a signal peptidase) by the host cell. For prokaryotic host cells that do not recognize and process the native antibody signal sequence, the signal sequence is substituted by a prokaryotic signal sequence selected.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

Suitable host cells for cloning or expressing the DNA are the prokaryote, yeast, or higher eukaryote cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1.982)); MRC 5 cells; FS4 cells; and a human hepatoma line (Hep G2).

Host cells are transformed with the above-described expression or cloning vectors for anti-rotavirus antibody production and cultured in conventional nutrient media modified as appropriate for inducing promoters, selecting transformants, or amplifying the genes encoding the desired sequences.

In some embodiments of the invention, the provided human antibody variable regions and/or CDR regions are used in a screening method to select for antibodies optimized for affinity, specificity, and the like. In such screening methods, random or directed mutagenesis is utilized to generate changes in the amino acid structure of the variable region or regions, where such variable regions will initially comprise one or more of the provided CDR sequences, e.g. a framework variable region comprising CDR1, CDR2, CDR3 from the heavy and light chain sequences provided herein.

These mutated variable region sequences, which are optionally combined with a second variable region sequence, i.e. V_{H}VL, with constant regions, as a fusion protein to provide for display, etc., as known in the art. Methods for selection of antibodies with optimized specificity, affinity, etc., are known and practiced in the art, e.g. including methods described by Presta (2006) Adv Drug Deliv Rev. 58(5-6):640-56; Levin and Weiss (2006) Mol Biosyst. 2(1):49-57; Rothe et al. (2006) Expert Opin Biol Ther. 6(2):177-87; Ladner et al. (2001) Curr Opin Biotechnol. 12(4):406-10; Amstutz et al. (2001) Curr Opin Biotechnol. 12(4):400-5; Nakamura and Takeo (1998) J Chromatogr B Biomed Sci Appl. 715(1):125-36 each herein specifically incorporated by reference for teaching methods of mutagenesis selection. Such methods are exemplified by Wu et al. (2005) J. Mol. Biol. (2005) 350, 126-144.

Such screening methods may involve mutagenizing a variable region sequence comprising one or more CDR sequences set forth herein; expressing the mutagenized sequence to provide a polypeptide product; contacting the polypeptide with an rotavirus antigen; identifying those polypeptide having the desired antigen affinity or specificity.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Formulations

The antibody formulations of the present invention may be used to treat the various rotavirus associated diseases as described herein. In some embodiments, the recipient is at a high risk of infection.

The antibody formulation is administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the antibody formulation is suitably administered by pulse infusion, particularly with declining doses of the antibody.

For the prevention or treatment of disease, the appropriate dosage of antibody will depend on the type of disease to be treated, the severity and course of the disease, whether the antibody is administered for preventive purposes, previous therapy, the patient's clinical history and response to the antibody, and the discretion of the attending physician. The antibody is suitably administered to the patient at one time or over a series of treatments.

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is one or more antibodies in a formulation of the invention as described above. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Therapeutic formulations comprising one or more antibodies of the invention are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to reduce virus titer in an infected individual.

The therapeutic dose may be at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. It will be understood by one of skill in the art that such guidelines will be adjusted for the molecular weight of the active agent, e.g. in the use of antibody fragments, or in the use of antibody conjugates. The dosage may also be varied for localized administration, or for systemic administration, e.g. i.m., i.p., i.v., and the like.

The antibody need not be, but is optionally formulated with one or more agents currently used to prevent or treat rotavirus infection. These are generally used in the same dosages and with administration routes as used hereinbefore or about from 1 to 99% of the heretofore employed dosages.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The formulation herein may also contain more than one active compound as necessary for the particular indication being treated, preferably those with complementary activities that do not adversely affect each other. For example, it may be desirable to further provide an antiviral agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

A pharmaceutically effective dose is that dose required to prevent, inhibit the occurrence, or treat (alleviate a symptom at least to some extent) of a disease state, e.g. to reduce virus titer in an infected individual. The pharmaceutically effective dose depends on the type of disease, the composition used, the route of administration, the type of subject being treated, subject-dependent characteristics under consideration, concurrent medication, and other factors that those skilled in the medical arts will recognize. Generally, an amount between 0.1 mg/kg and 100 mg/kg body weight/day of active ingredients is administered.

Oral administration can be accomplished using pharmaceutical compositions containing an agent of interest formulated as tablets, lozenges, aqueous or oily suspensions, dispersible powders or granules, emulsion, hard or soft capsules, or syrups or elixirs. Such oral compositions can contain one or more such sweetening agents, flavoring agents, coloring agents or preservative agents in order to provide pharmaceutically elegant and palatable preparations. Tablets, which can be coated or uncoated, can be formulated to contain the active ingredient in admixture with non-toxic pharmaceutically acceptable excipients, e.g., inert diluents; such as calcium carbonate, sodium carbonate, lactose, calcium phosphate or sodium phosphate; granulating and disintegrating agents, for example, corn starch, or alginic acid; binding agents, e.g., starch, gelatin or acacia; and lubricating agents, for example magnesium stearate, stearic acid or talc. Where a coating is used, the coating delay disintegration and absorption in the gastrointestinal tract and thereby provide a sustained action over a longer period.

Where the formulation is an aqueous suspension, such can contain the active agent in a mixture with a suitable excipient(s). Such excipients can be, as appropriate, suspending agents (e.g., sodium carboxymethylcellulose, methylcellulose, hydropropyl-methylcellulose, sodium alginate, polyvinylpyrrolidone, gum tragacanth and gum acacia); dispersing or wetting agents; preservatives; coloring agents; and/or flavoring agents.

Suppositories, e.g., for rectal administration of agents, can be prepared by mixing the agent with a suitable non-irritating excipient that is solid at ordinary temperatures but liquid at the rectal temperature and will therefore melt in the rectum to release the drug. Such materials include cocoa butter and polyethylene glycols.

Dosage levels can be readily determined by the ordinarily skilled clinician, and can be modified as required, e.g., as required to modify a subject's response to therapy. In general dosage levels are on the order of from about 0.1 mg to about 140 mg per kilogram of body weight per day. The amount of active ingredient that can be combined with the carrier materials to produce a single dosage form varies depending upon the host treated and the particular mode of administration. Dosage unit forms generally contain between from about 1 mg to about 500 mg of an active ingredient.

### Peptide Vaccine Compositions

The application discloses herein a method of inducing an immune response against a peptide corresponding to an epitope recognized by an antibody disclosed herein, including without limitation specific epitopes of the VP4, VP5*, or VP7, where the epitope is of sufficient length to provide for binding specificity substantially similar to the specificity of binding to the native protein, e.g. a peptide of at least 20 amino acids, at least 30 amino acids, at least 40 amino acids, at least 50 amino acids, at least 100 amino acids, at least 150 amino acids, at least 200 amino acids up to the full length of the protein, where the peptide may be a contiguous or non-contiguous sequence of an rotavirus protein.

A basis for heterotypic neutralizing reactivity to RV in humans at the individual immunoglobulin (Ig) molecule level is identified. In some embodiments a method of defining such activity is provided, comprising the steps of sorting single cells of intestinal RV-specific IgA⁺ antibody secreting cells, by contacting the cells with triple-layered RV particles conjugated to a detectable label, e.g. a fluorochrome suitable for sorting by flow cytometry. The immunoglobulin coding polynucleotides from such sorted cells are sequenced with an identifying barcode. The antibodies thus identified by sequences are tested for activity in RV neutralization in vitro against two or more different RV serotypes, where antibodies that neutralize multiple serotypes are defined as heterotypic antibodies. The methods are useful in providing detailed analysis of thre ability of an immunogen, e.g. a vaccine, to elicit a protective heterotypic response. Humans can circumvent the serotypic diversity of naturally circulating RV strains by expressing individual VP4 epitope-specific Ig molecules that mediate heterotypic neutralization. Characterization of the structural targets of these mAbs, and determination of the extent to which they arise following primary RV infection of children provide the basis for designing more effective RV vaccines.

Antigenic compositions are provided, which comprise all or a portion of a rotavirus protein in which specific highly immunodominant residues are masked or deleted, so as to generate an immune response to residues that are less immunodominant, but which are essential for virus function and therefore are less likely to be altered in virus escape mutation and selection. Alternatively antigenic compositions providing epitopes for heterotypic neutralizing antibodies are provided, which can be formulated alone or in combination with conventional vaccines. Antigens may comprise, without limitation, VP5* proteins, alone or in combination with an adjuvant. These antigens find use in screening assays, generation of monoclonal antibodies, and in vaccines. Such formulations may comprise, without limitation, live attenuated formulation containing known heterotypic neutralizing epitopes (and excluding known homotypic neutralizing epitopes); and/or epitope immunogens with known heterotypic neutralizing epitopes or overlapping neutralizing epitopes. These novel vaccines/immunogens could be used in combination with current formulations, for example in a prime boost strategy to enhance immunity in children and infants who do not respond to the current, licensed vaccines or formulations alone. The formulations of the invention may find particular benefit in providing improved protective immunity in regions of the world with the highest RV disease burden and lowest vaccine efficacy observed in several clinical trials of the current licensed RV vaccines.

In some embodiments of the invention, a modified rotavirus VP4, including a VP5* fragment, or VP7 polypeptide is provided, which provides for enhanced heterotypic immune responsiveness In other embodiments, a polynucleotide encoding such a modified rotavirus polypeptide is provided. The polypeptide and/or the nucleic acid can be used in the formulation of a vaccine, e.g. a virus-like particle, a recombinant protein vaccine which can be formulated with an adjuvant, a vector vaccine, and the like. In some embodiments, a vaccine formulation comprising a polypeptide or a polynucleotide of the invention is provided.

In some embodiments, portions of the rotavirus protein or live-attenuated whole virus are provided as an immunogen known to stimulate heterotypic protective immunity in humans as determined by epitope mapping studies using these mAbs. All or a portion of the rotavirus protein is provided as an antigen, where specific highly immunodominant residues are masked, so as to allow for the generation of an immune response to residues that are less immunodominant, but which are essential for virus function and therefore are less likely to be altered. These antigens find use in screening assays, generation of monoclonal antibodies, and in vaccines. Peptides for immunization may be conjugated to a carrier molecule prior to administration to a subject.

Peptides can be produced using techniques well known in the art. Such techniques include chemical and biochemical synthesis. Examples of techniques for chemical synthesis of peptides are provided in Vincent, in Peptide and Protein Drug Delivery, New York, N.Y., Dekker, 1990. Examples of techniques for biochemical synthesis involving the introduction of a nucleic acid into a cell and expression of nucleic acids are provided in Ausubel, Current Protocols in Molecular Biology, John Wiley, and Sambrook, et al in Molecular Cloning, A Laboratory Manual, Cold Spring Harbor Laboratory Press, 1989.

In the methods disclosed herein, an immunologically effective amount of one or more immunogenic polypeptides, which may be conjugated to a suitable carrier molecule, is administered to a patient by successive, spaced administrations of a vaccine, in a manner effective to result in an improvement in the patient's condition.

In an exemplary embodiment, immunogenic polypeptides are coupled to one of a number of carrier molecules, known to those of skill in the art. A carrier protein must be of sufficient size for the immune system of the subject to which it is administered to recognize its foreign nature and develop antibodies to it.

In some cases the carrier molecule is directly coupled to the immunogenic peptide. In other cases, there is a linker molecule inserted between the carrier molecule and the immunogenic peptide. For example, the coupling reaction may require a free sulfhydryl group on the peptide. In such cases, an N-terminal cysteine residue is added to the peptide when the peptide is synthesized. In an exemplary embodiment, traditional succinimide chemistry is used to link the peptide to a carrier protein. Methods for preparing such peptide:carrier protein conjugates are generally known to those of skill in the art and reagents for such methods are commercially available (e.g., from Sigma Chemical Co.). Generally about 5-30 peptide molecules are conjugated per molecule of carrier protein.

Exemplary carrier molecules include proteins such as keyhole limpet hemocyanin (KLH), bovine serum albumin (BSA), flagellin, influenza subunit proteins, tetanus toxoid (TT), diphtheria toxoid (DT), cholera toxoid (CT), a variety of bacterial heat shock proteins, glutathione reductase (GST), or natural proteins such as thyroglobulin, and the like. One of skill in the art can readily select an appropriate carrier molecule. In some cases, the carrier molecule is a non-protein, such as Ficoll 70 or Ficoll 400 (a synthetic copolymer of sucrose and epichlorohydrin), a polyglucose such as Dextran T 70.

Another category of carrier proteins is represented by virus capsid proteins that have the capability to self-assemble into virus-like particles (VLPs). Examples of VLPs used as peptide carriers are hepatitis B virus surface antigen and core antigen, hepatitis E virus particles, polyoma virus, and bovine papilloma virus.

A peptide vaccine composition may comprise single or multiple copies of the same or different immunogenic peptide, coupled to a selected carrier molecule. In one aspect of this embodiment, the peptide vaccine composition may contain different immunogenic peptides with or without flanking sequences, combined sequentially into a polypeptide and coupled to the same carrier. Alternatively, immunogenic peptides, may be coupled individually as peptides to the same or a different carrier, and the resulting immunogenic peptide-carrier conjugates blended together to form a single composition, or administered individually at the same or different times.

In general, peptide vaccine compositions are administered with a vehicle. The purpose of the vehicle is to emulsify the vaccine preparation. Numerous vehicles are known to those of skill in the art, and any vehicle which functions as an effective emulsifying agent finds utility in the present invention. To further increase the magnitude of the immune response resulting from administration of the vaccine, an immunological adjuvant may be included in the vaccine formulation. Exemplary adjuvants known to those of skill in the art include water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs.

Suitable pharmaceutically acceptable carriers for use in an immunogenic proteinaceous composition of the invention are well known to those of skill in the art. Such carriers include, for example, phosphate buffered saline, or any physiologically compatible medium, suitable for introducing the vaccine into a subject.

Numerous drug delivery mechanisms known to those of skill in the art may be employed to administer the immunogenic peptides of the invention. Controlled release preparations may be achieved by the use of polymers to complex or absorb the peptides or antibodies. Controlled delivery may accomplished using macromolecules such as, polyesters, polyamino acids, polyvinyl pyrrolidone, ethylenevinylacetate, methylcellulose, carboxymethylcellulose, or protamine sulfate, the concentration of which can alter the rate of release of the peptide vaccine.

In some cases, the peptides may be incorporated into polymeric particles composed of e.g., polyesters, polyamino acids, hydrogels, polylactic acid, or ethylene vinylacetate copolymers. Alternatively, the peptide vaccine is entrapped in microcapsules, liposomes, albumin microspheres, microemulsions, nanoparticles, nanocapsules, or macroemulsions, using methods generally known to those of skill in the art.

The vaccine of the present invention can be administered to patient by different routes such as intravenous, intraperitoneal, subcutaneous, intramuscular, or orally. A preferred route is intramuscular or oral. Suitable dosing regimens are preferably determined taking into account factors well known in the art including age, weight, sex and medical condition of the subject; the route of administration; the desired effect; and the particular conjugate employed (e.g., the peptide, the peptide loading on the carrier, etc.). The vaccine can be used in multi-dose vaccination formats.

It is expected that a dose would consist of the range of to 1.0 mg total protein. In an embodiment of the present invention the range is 0.1 mg to 1.0 mg. However, one may prefer to adjust dosage based on the amount of peptide delivered. In either case these ranges are guidelines. More precise dosages should be determined by assessing the immunogenicity of the conjugate produced so that an immunologically effective dose is delivered. An immunologically effective dose is one that stimulates the immune system of the patient to establish a level immunological memory sufficient to provide long term protection against disease caused by infection with rotavirus. The conjugate is preferably formulated with an adjuvant.

The timing of doses depend upon factors well known in the art. After the initial administration one or more booster doses may subsequently be administered to maintain antibody titers. An example of a dosing regime would be a dose on day 1, a second dose at or 2 months, a third dose at either 4, 6 or 12 months, and additional booster doses at distant times as needed.

The vaccine formulation is administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the vaccine formulation is suitably administered by pulse infusion, particularly with declining doses of the vaccine.

For the prevention or treatment of disease, the appropriate dosage of vaccine will depend on the type of disease to be treated, the severity and course of the disease, whether the vaccine is administered for preventive purposes, previous therapy, the patient's clinical history and response to the vaccine, and the discretion of the attending physician. The vaccine is suitably administered to the patient at one time or over a series of treatments.

In another embodiment of the invention, an article of manufacture containing materials useful for the vaccination described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is one or more antibodies in a formulation of the invention as described above. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Therapeutic formulations are prepared for storage by mixing the vaccine having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The vaccine composition will be formulated, dosed, and administered in a fashion consistent with good medical practice. The “therapeutically effective amount” of the vaccine to be administered will be governed by clinical considerations, and is the minimum amount necessary to reduce virus titer in an infected individual.

One may adjust dosage based on the amount of peptide delivered. An immunologically effective dose is one that stimulates the immune system of the patient to establish a level immunological memory sufficient to provide long term protection against disease caused by infection with rotavirus. More precise dosages should be determined by assessing the immunogenicity of the vaccine produced so that an immunologically effective dose is delivered.

The therapeutic dose may be at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. It will be understood by one of skill in the art that such guidelines will be adjusted for the molecular weight of the active agent, e.g. in the use of vaccine fragments, or in the use of vaccine conjugates. The dosage may also be varied for localized administration, or for systemic administration, e.g. i.m., i.p., i.v., and the like.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Where the formulation is an aqueous suspension, such can contain the active agent in a mixture with a suitable excipient(s). Such excipients can be, as appropriate, suspending agents (e.g., sodium carboxymethylcellulose, methylcellulose, hydropropyl-methylcellulose, sodium alginate, polyvinylpyrrolidone, gum tragacanth and gum acacia); dispersing or wetting agents; preservatives; coloring agents; and/or flavoring agents.

Suppositories, e.g., for rectal administration of agents, can be prepared by mixing the agent with a suitable non-irritating excipient that is solid at ordinary temperatures but liquid at the rectal temperature and will therefore melt in the rectum to release the drug. Such materials include cocoa butter and polyethylene glycols.

In one aspect, the invention provides a means for classifying the immune response to peptide vaccine, e.g., 9 to 15 weeks after administration of the vaccine; by measuring the level of antibodies against the immunogenic peptide of the vaccine.

The invention now being fully described, it will be apparent to one of ordinary skill in the art that various changes and modifications can be made without departing from the spirit or scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1

### VP4- and VP7-Specific Antibodies Mediate Heterotypic Immunity to Rotavirus in Humans

Homotypic and heterotypic neutralization domains on both VP4 and VP7 have been identified using murine monoclonal antibodies (mAbs) elicited following parenteral hyper-immunization. The near-atomic structure of neutralizing antigenic epitopes on VP4 and VP7 from animal and, to a limited degree, human RVs have been elucidated using murine mAbs. To date, the majority of isolated VP7-specific neutralizing mAbs have been serotype specific; however, the epitope containing amino acid (AA) 94 of VP7 has been shown in one case to be targeted by a heterotypic neutralizing IgM mAb as well as homotypic mAbs. Based on limited neutralizing murine mAb studies, VP5*, the carboxy-terminal trypsin cleavage fragment of VP4, appears to be the target of murine mAbs with heterotypic specificity. In contrast, murine mAbs to VP8*, the amino terminal trypsin cleavage fragment of VP4, are primarily serotype specific.

Epidemiologic studies and clinical trials worldwide clearly demonstrate that heterotypic protective immunity is generated following a single symptomatic or asymptomatic RV infection or immunization with the monovalent, monotypic Rotarix (G1P[8]) or Rotavac (Bharat Biotech, G9P[11]) vaccines; however, the molecular basis for this broad protective immunity following monotypic exposure is unknown. One hypothesis is that individual anti-VP4 and anti-VP7 Abs with heterotypic cross-reactivity are generated following monotypic RV natural infection or immunization and that these Abs mediate broad-based protective immunity following re-exposure to new RV serotypes. This hypothesis is supported by the comparable efficacy of the monovalent Rotarix and the pentavalent Rotateq vaccines. Rotateq contains five live, reassortant RVs each expressing serotypically distinct RV antigens, and Rotarix contains only a single RV strain. An alternate hypothesis is that heterotypic immunity is mediated by an array of individual Ab molecules, each with restricted neutralizing specificity against a single RV serotype. Only one study has directly examined the serotypic specificity of human anti-VP4 and anti-VP7 mAbs. Three human single chain mAbs were generated from a pooled bone marrow-derived phage display library; two VP4 mAbs had heterotypic specificities, whereas the single VP7 mAb isolated was homotypic. A third, not mutually exclusive, hypothesis suggests that Abs to common antigens on the RV virion (such as VP6) might provide protection in vivo even though these Abs lack neutralizing activity in traditional in vitro functional assays.

In this study, we combined single-cell sorting of RV TLP-binding intestinal IgA⁺ antibody secreting cells (ASCs) with barcode-based next-generation sequencing of paired IGHV and IGLV Ab genes to identify RV-specific mAbs from the intestines of RV-experienced adults. We demonstrate that individual mAbs specific for VP7 and VP4 (VP5*) mediate potent heterotypic neutralizing activity in vitro and in vivo whereas the multiple mAbs that bind VP8* appear to be functionally inactive in a traditional in vitro neutralization assay. These findings reveal a molecular basis for the broad-based heterotypic protection observed in humans against the multitude of serotypically distinct RV strains circulating worldwide. Our data also identify target antigens that guide the design of more effective, next-generation RV vaccines.

### Results

Isolation of Rotavirus TLP-Specific Intestinal B Cells by Flow Cytometry.

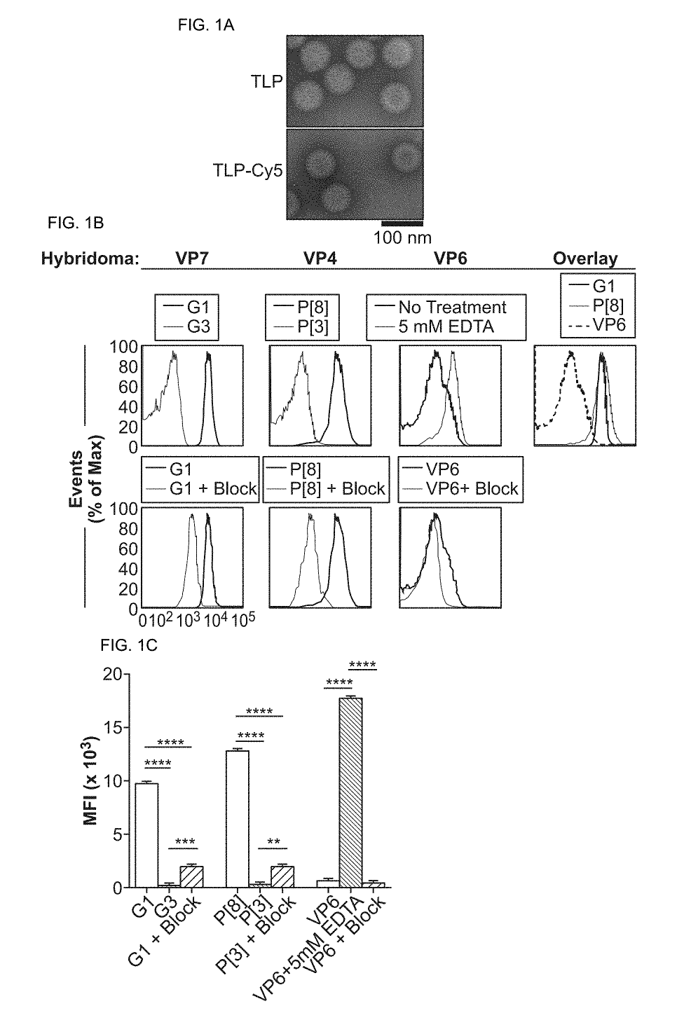

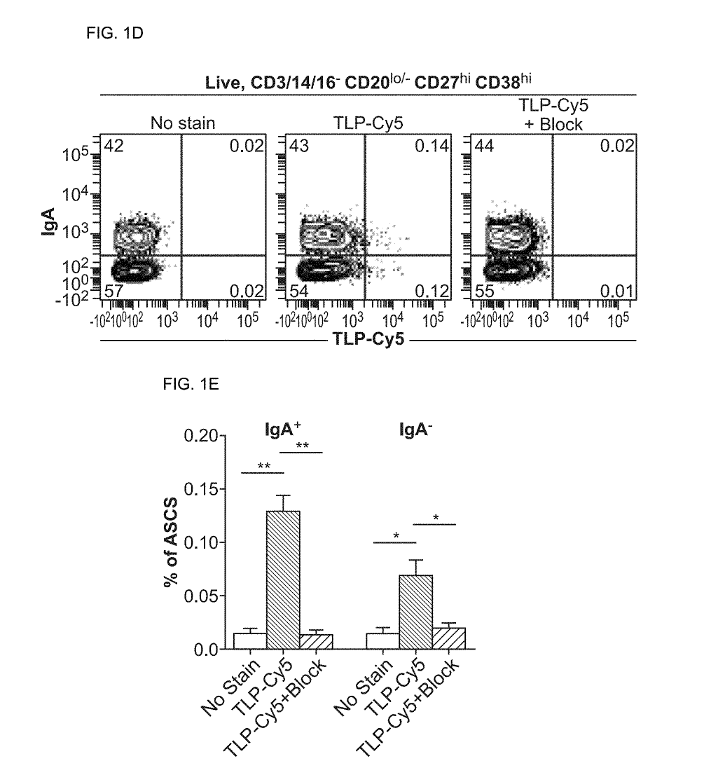

Highly purified triple layered particles (TLPs) (CDC-9, G1P[8]) were amine-labeled with Cy5, and the structural integrity of the labeled conjugates was confirmed by electron microscopy (FIG. 1A). The binding of TLP-Cy5 to B cells expressing surface VP4- or VP7-specific Ig was assessed by FACS in staining and blocking experiments using mouse hybridoma cells with previously characterized specificity for VP6 and for genotypically or serotypically distinct VP4s (P[8] or P[3]) and VP7s (G1 or G3). CDC-9 TLP-Cy5 stained G1- and P[8]-specific hybridomas (mean fluorescence intensity (MFI)±SD) for G8 and P[8] of 9800±71, 12801±282, respectively) but did not stain G3- or P[3]-specific hybridomas (FIG. 1B, C). Pre-incubation of G1- or P[8]-specific hybridomas with unlabeled CDC-9 TLPs reduced TLP-Cy5 binding to both hybridoma cell lines (G1 by 5-fold, P[8] by 7-fold). The binding of CDC-9 TLPs-Cy5 to VP6-specific hybridomas (MFI±SD, 737±89) was lower than that observed to G1-specific hybridomas (by 13 fold) and to P[8]-specific hybridomas (by 17 fold). Treatment of the TLP-Cy5 with EDTA prior to staining, which dissociates the two outer capsid proteins from triple layered virion particles, resulted in a 24-fold increase in binding to VP6-specific hybridomas (FIG. 1C). Pre-incubation of VP6-specific hybridomas with unlabeled TLPs reduced TLP-Cy5 staining on VP6-specific hybridomas by only 2 fold. Once the specificity of the TLP-Cy5 for these well-characterized murine hybridomas was established, we proceeded to stain isolated human intestinal B cells with the RV TLP-Cy5 preparation. Unstained control cells were used to identify TLP-specific IgA⁺ and IgA⁻ ASCs. Cold blocking with unlabeled TLPs reduced TLP-Cy5 staining on total intestinal B cells by 10 fold in IgA⁺ ASCs and by 4 fold in IgA ASCs (FIG. 1D, E).

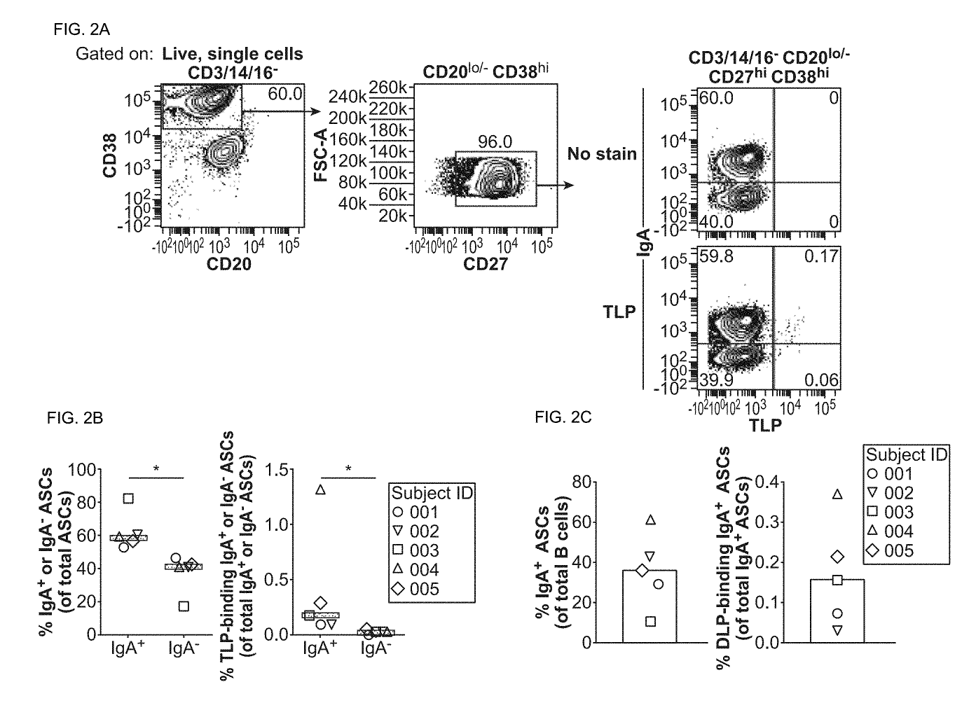

The CDC-9 TLP-Cy5 particles were then used to isolate RV-reactive B cells from proximal jejunum resections obtained from five adult patients undergoing bariatric surgery. ASCs, which include both plasmablasts and plasma cells, were identified by flow cytometry by gating on live, single CD3/14/16⁻ CD20^{lo} CD27^{hi} CD38^{hi} cells. IgA⁺ ASCs were identified based on surface Ig expression. TLP-binding IgA⁺ ASCs were defined using gating based on unstained ASCs (FIG. 2A). The majority of intestinal ASCs in the five adult subjects were IgA⁺ (median frequency 59.4%, range 53.4-82.5%). Intestinal IgA ASCs were detected at a median frequency of 40.6% (range 17.5-46.7%). Within the ASCs, most RV TLP-binding reactivity was detected among IgA⁺ ASCs (median frequency 0.13%, range 0.09-1.30%). Some TLP-binding reactivity was also observed among IgA ASCs (median frequency 0.01%, range 0.00-0.04%) (FIG. 2B). An ELISPOT assay with RV double-layered protein capsids (DLPs) as the detecting antigen, which primarily detects Abs directed at the inner capsid VP6 protein, confirmed that the five selected donors had readily detectable levels of functional Ab-secreting RV-specific B cells in their proximal small bowel. The median frequency of total IgA⁺ ASCs as a proportion of total intestinal B cells was 35.2% (range 10.0-61.6%). The median frequency of VP6-specific IgA⁺ ASCs as a proportion of total IgA⁺ ASCs was 0.16% (range 0.03-0.37%) (FIG. 2C).

Molecular Features of RV TLP-Reactive IgA⁺ Intestinal ASCs.

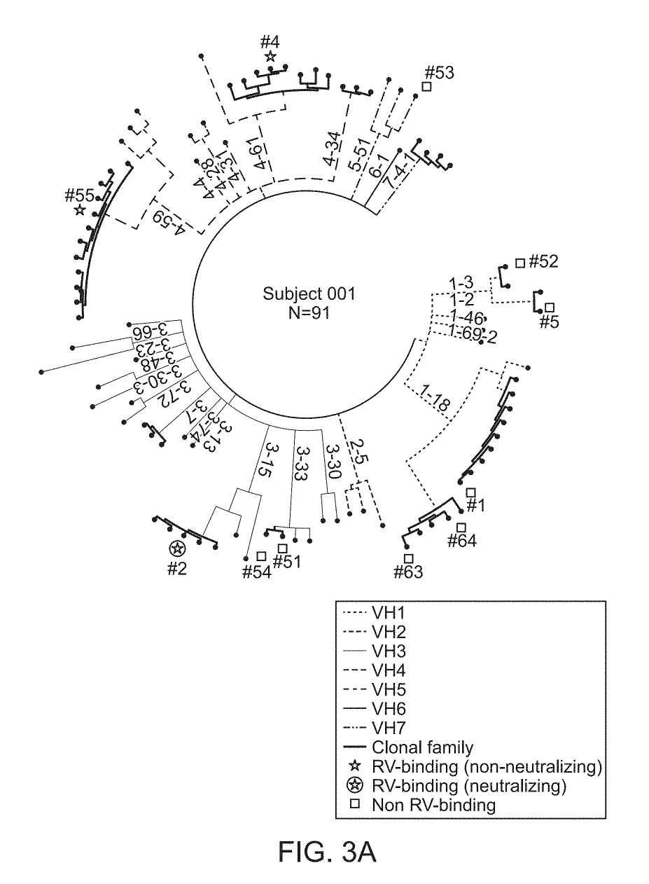

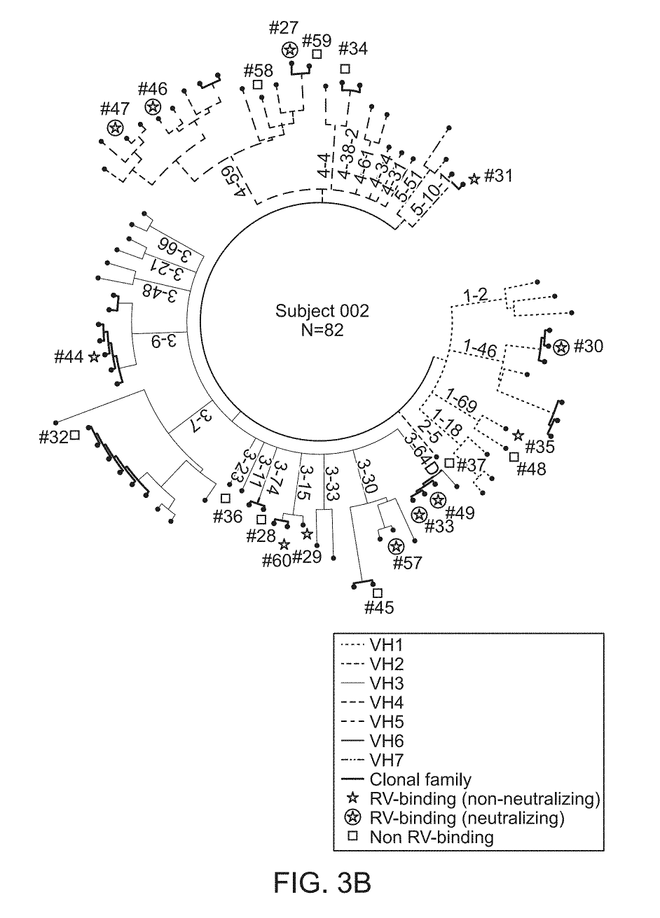

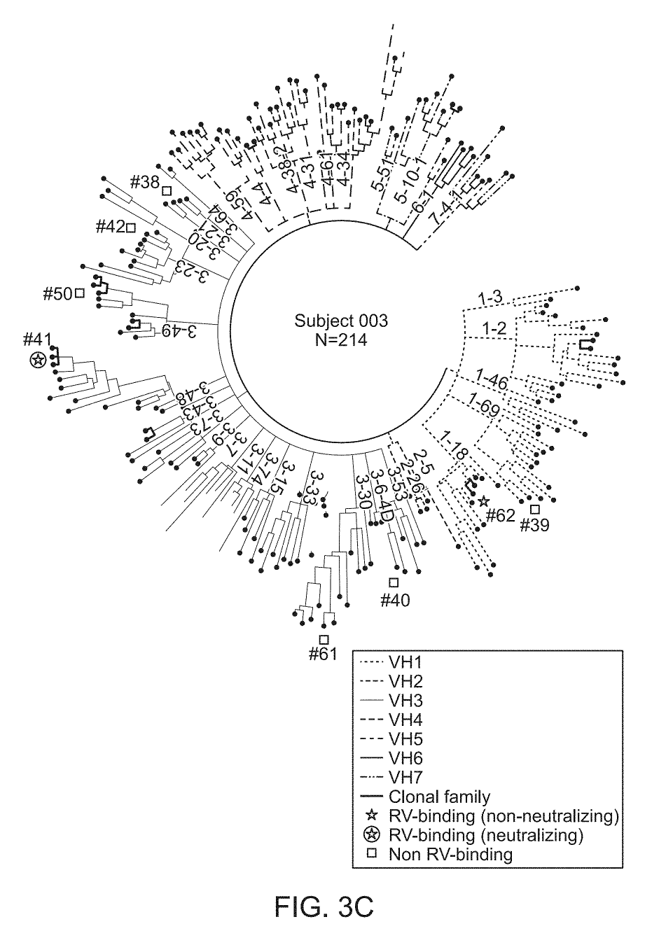

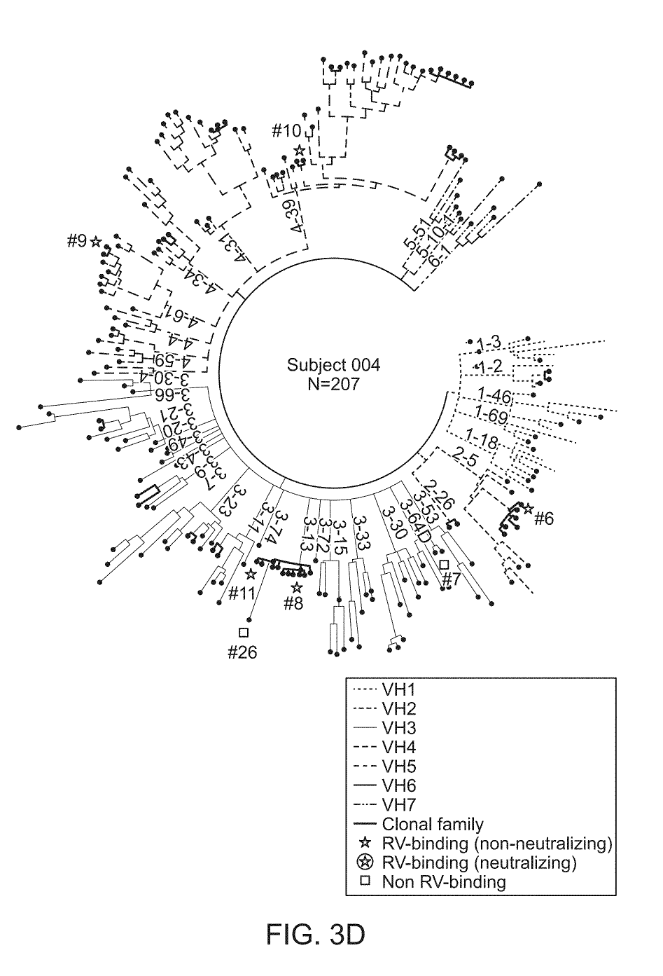

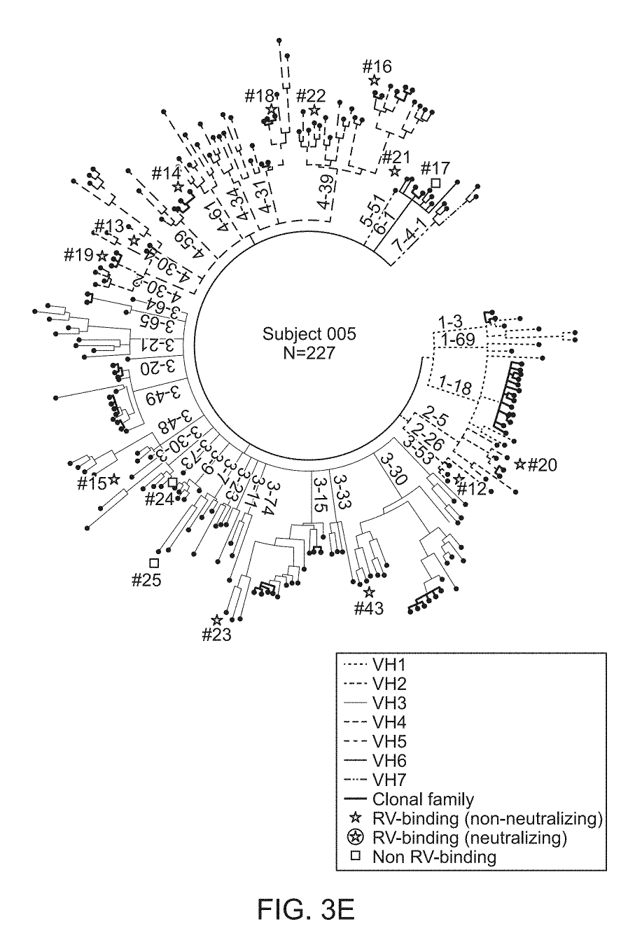

Intestinal IgA⁺ ASCs were single-cell sorted, and the cDNAs from individual Ig mRNAs were synthesized incorporating a well- and plate-specific barcode as previously described. The unique barcodes were used to specifically pair native heavy and light chain Ab sequences derived from the individual B cell in the specific well. Sequences of the specific heavy and light chains from each Ab were then used to construct dendrograms of each donor's IgA⁺ ASC heavy and light chain repertoire to visualize the individual Abs using identical germline IGHV and IGLV genes (FIG. 3). Clonal families were defined as having the same V and J gene segment usage for both heavy- and light-chain and CDR3 amino acid Levenshtein distances ≤3 for both heavy- and light-chain. IGHV D alleles were not used in the clonal family assignments due to their short length (<20 AA) and high mutation rates. Ab sequences that used V and J gene segments for heavy- and light-chains that were not used by any other Ab in the populations of single cells sampled from each individual and that had CDR3 amino acid Levenshtein distance >3 for both heavy- and light-chain were termed “singletons”.

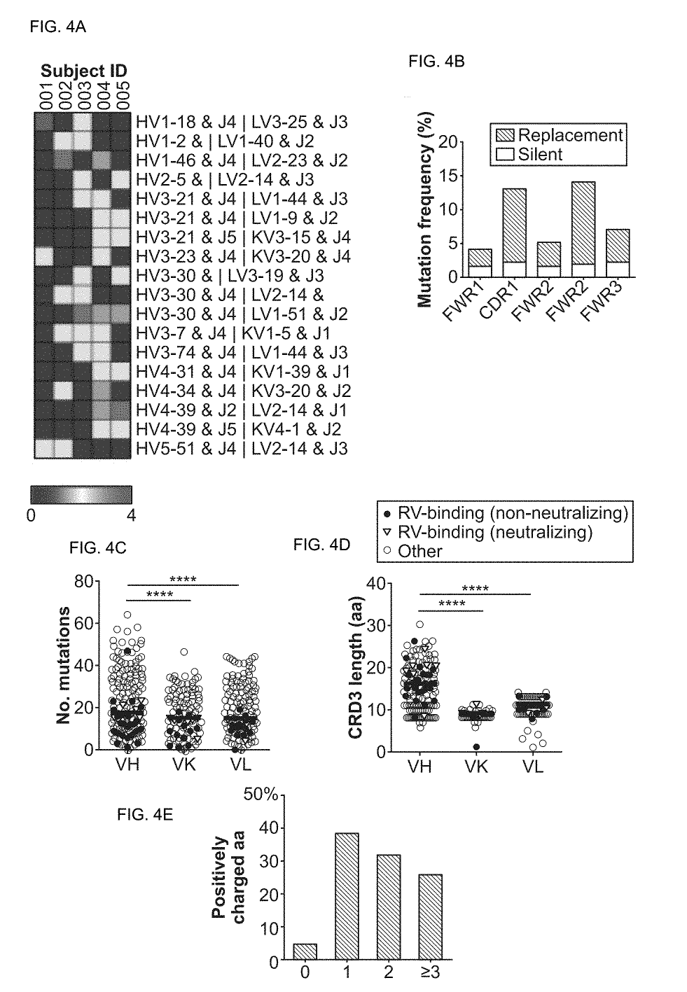

A total of 821 paired IGHV and IGLV sequences were analyzed from the five donors; the median number of paired Ab sequences recovered per donor was 207 (range 82-227). Clonally related Abs were identified at an overall frequency of 29% of all paired sequences from all donors (9-60% in individual donors) (FIG. 3). The median number of clonal families per subject was 12 (range 8-16), with a range of 2-14 Abs per clonal family. The frequency of combinatorial IGVH-IGVL gene segment usage among all donors was analyzed. The majority (613) of VH-VL combinations were unique to individual donors. Sixteen (2.6%) IGVH-IGVL gene combinations were detected in two donors, and two (0.3%) sequences were detected in three donors. No combinations were detected in more than three donors (FIG. 4A).

All Abs showed high numbers of somatic mutations and replacements to silent mutations in complementarity determining regions (CDR) 1 and 2 compared with those in framework regions (FWR) 1, 2, and 3, consistent with antigen-mediated selection in their IGH and/or IGK and IGL chain genes (FIG. 4B). V segments of IgA⁺ ASCs carried a median of 19 (range 2-65) mutations. Median numbers of mutations in K and L segments were 15 (range 1-41) and 14 (range 1-47), respectively (FIG. 4C). The median CDR3 length across all Abs was 16 amino acids for VH (range 6-30), nine for VK (range 1-11), and 11 for VL (range 1-14) (FIG. 4D, E). Most Abs contained two or more positively charged amino acids in CDRH3 indicative of high-affinity binding.

Specificity of Human mAbs.

Thirty-five pairs of IGHV and IGLV sequences from various clonal families were selected for expression as recombinant mAbs for characterization of RV binding and neutralization reactivity, and 27 VH/VL pairs encoding singletons were also expressed for comparison (numbered in FIG. 3). Each mAb was first screened to determine its binding reactivity against recombinant VP2-eGFPN/P6 DLP virus-like particles (VLPs), Wa strain RV-derived DLPs, and replication competent CDC-9 TLPs (G1P[8]) by ELISA. RV-directed VP4- or VP7-specific mAbs were defined as those mAbs that bound to TLPs but not to purified DLPs or recombinant VP2/VP6 DL-VLPs. VP6-specific mAbs were defined as those that bound specifically to purified DLPs and/or recombinant VP2/VP6 DL-VLPs whether or not they bound to TLPs. Table 1 summarizes the protein specificities of the recombinant mAbs. Thirty-three of the 62 expressed mAbs bound to TLPs but not to DLPs, and hence were presumed to be either VP4- or VP7-specific. One mAb bound to both TLPs and DLPs and was presumed to be VP6 specific. Therefore, 55% of the flow cytometry-selected individual ASCs encoded Ab sequences were RV-specific. Of the 34 RV specific mAbs, 23 were derived from clonal families and 11 were classified as singletons (FIG. 3 and Table 1). Among the combinations of VH-VL gene segments that were shared across donors, none were present in mAbs with confirmed RV-binding specificity, while two shared combinations of VH-VL were present in mAbs that did not bind RV (FIG. 4A). The remaining VH-VL gene combinations shared across different donors were not expressed and thus their RV binding specificity was not determined in this study.

Next, the binding reactivity of the VP4/VP7 RV reactive recombinant mAbs against selected human and animal origin RV strains of different G and P types was examined by a variety of assays including immunostaining of RV-infected MA-104 cells, immunostaining of Sf9 cells infected with recombinant baculovirus (BV) that expressed specific RV proteins, ELISA binding to the recombinant VP8* or VP5*, and immunoprecipitation of selected recombinant RV proteins expressed in vitro (Tables 1, 2, and 3). All 33 VP4- or VP7-specific mAbs bound to Wa (G1P[8])-infected MA104 cells (Table 2). Twenty-six of these mAbs also bound to DS-1 (G2P[4])-infected cells. Some mAbs displayed binding reactivity to cells infected with non-human RV strains: three to RRV, ST3, NCDV, and OSU; two to ST3; one to ST3, NCDV, and OSU; one to NCDV and OSU; one to ST3, RRV, and OSU; and one to ST3, RRV, and NCDV (Table 3). Hence, the VP4/VP7-specific mAbs examined displayed varying but, in most cases, substantial degrees of heterotypic reactivity as measured by binding to cells infected with multiple RV serotypes. However, six VP4/VP7-specific mAbs bound only to cells infected with Wa RV including three of the four VP7 mAbs (Table 3).

To determine the binding specificity of the mAbs at the individual RV protein level, immunostaining was performed using Sf9 cells infected with recombinant BVs expressing RV VP4 (KU P[8], DS1 P[4], 1076 P[6]), VP7 (Wa G1), or VP6 (RRV) (Table 3). Of the 33 isolated mAbs that were VP4/VP7-specific as determined by TLP binding, 29 bound to recombinant VP4, and only four bound to recombinant VP7 (Table 1, Table 3). Among the 29 VP4-specific mAbs, five bound to all three recombinant baculovirus-expressed VP4 proteins, five bound only to P[8], one bound only to P[4], while 18 bound to P[4] and P[8] (Table 3). Only the one DLP-binding mAb bound to recombinant VP6. Further resolution of the binding specificity of VP4-specific mAbs revealed that the great majority (22 of 29) bound to recombinant bacterially expressed VP8*, the amino terminal trypsin cleavage fragment of VP4, as determined by ELISA (Table 3) and almost all of these bound only to the VP4s expressed by Wa alone or by Wa and DS1. Five of the VP4-specific mAbs bound specifically to recombinant VP5*, the carboxy terminal stalk region of VP4. These five mAbs did not bind to recombinant VP8* (Table 1, Table 3). VP5* binding was assessed by immunoprecipitation of in vitro translated VP5* as previously described. The binding site specificity of two VP4 specific mAbs could not be determined using these strategies (Table 1).

VP4- and VP7-Specific Intestinal-Derived mAbs Display Neutralizing Activity In Vitro.

In vitro neutralization capacities of the VP4 and VP7 binding recombinant mAbs were assessed in assays using the Wa and CDC-9 RV strains (G1P[8]), three VP7 mono-reassortants including D×RRV (G1P[3], DS1×RRV (G2P[3]) and ST3×RRV (G4P[3], and a set of serotypically distinct animal and human RV strains including DS1 (G2P[4]), RRV (G3P[3]), ST3 (G4P[6]), OSU (G5P[7]), NCDV (G6P[1]), UK (G6P[5]), 69M (G8P[10]), 116E (G9P[11]), WI61 (G9P[8]), and L26 (G12P[4]) (Table 2, Table S1 and data not shown). Data are summarized in Tables 1 and 2. Nine of the 34 (26%) RV-reactive mAbs isolated from the five adult subjects neutralized one or more of these RV strains in vitro. Three of the nine mAbs (mAb #27, #46 and #57) were VP7-specific as determined by their ability to neutralize the G1 VP7 monoreassortant D×RRV but not the G3 parental RRV strain and by their specific immunostaining of Sf9 cells infected with BVs expressing Wa VP7. Another six were VP4-specific as determined by specific binding assays to various forms of recombinant VP4. Five of the six VP4-specific neutralizing mAbs bound to recombinant VP5* but did not bind recombinant VP8*. One of the six VP4-specific neutralizing mAbs did not bind to either recombinant VP5* or VP8*. Surprisingly, neutralizing capacity was not detected in any of the 22 VP4-directed mAbs that bound to a recombinant VP8* fragment either in traditional neutralization assay using MA104-infected cells or in an experimental neutralization assay using primary human small intestinal organoids as the target cell substrate.

In assessment of the serotypic specificities, we defined homotypic neutralizing mAbs as mAbs for which the neutralization activity, defined by minimum neutralization concentration of the mAb, to a single serotype (G or P) was >10 fold higher than that to other serotypes. Heterotypic mAbs were defined as those with minimum neutralization concentration within 10-fold for two or more distinct serotypes. Based on these criteria, three of the nine neutralizing Abs were homotypic: VP4 (VP5*)-specific mAb #33 (P[8]) and VP7-directed mAbs #27 (G1) and #46 (G1). Six of the nine neutralizing Abs were heterotypic: VP5*-specific mAb #2, P[4] and P[8]; mAb #30, P[8], P[4], and P[6]; mAb #41, P[8], P[6], P[4], and P[3]; mAb #49, P[8], P[1], and P[3]; VP4-specific mAb #47, P[8] and P[4]; and VP7-directed mAb #57, G1, G2, and G3 (Table 2). mAb #49 demonstrated a low level of neutralizing activity with the highest minimum neutralizing concentration against a human RV strain at 39.1 ng/ml against Wa. Human RV strains 69M (G8P[10]), 116E (G9P[11]), and #321 (G10P[11]) were not neutralized by any of the mAbs at concentrations up to 625 ng/ml. Analysis of the molecular features of the nine neutralizing mAbs revealed distinct VH and VL gene segment usages as well as distinct CDRH3 AA sequences and lengths (Table 3, FIG. 4B-D). Six of the nine neutralizing mAbs belonged to clonal families and three were singletons (FIG. 3).

VP4- and VP7-Specific Intestinal mAbs Display Both Homotypic and Heterotypic Neutralizing Activity In Vivo.

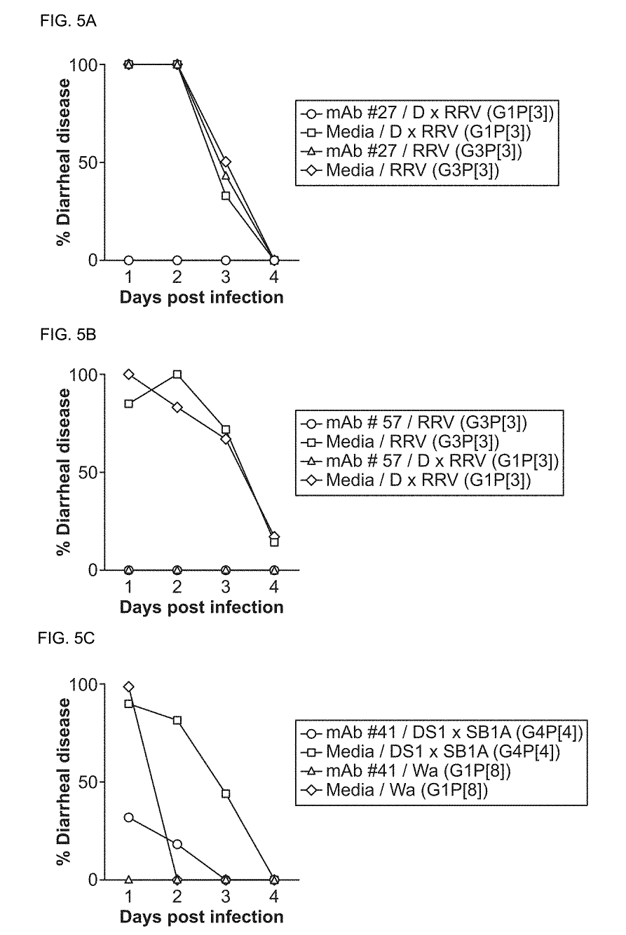

The ability of mAbs to protect against RV-induced diarrheal disease in vivo was examined using rhesus RRV (G3P[3]), a human RV VP7-RRV mono-reassortant D×RRV (G1P[3]), the monoreassortant DS1×SB1A (G4P4), and Wa RV (G1P8) as challenge strains. VP7-specific mAb #27 (G1 specific), when co-incubated with RRV (G3 serotype) or D×RRV (G1 serotype) and then administered orally at a dose of 10⁶ PFU to 5-day-old suckling 129/Sv mice, prevented the G1 D×RRV-induced but not the G3 RRV-induced diarrheal disease (FIG. 5A). VP7-directed mAb #57, which neutralized both G1 and G3 RV strains in vitro, protected against both RRV- and D×RRV-induced diarrhea at an efficacy of 100% (FIG. 5B). mAb #41, which is directed at VP5* and neutralized both P[4] and P[8] RV strains in vitro, had a protective efficacy of 67% against the P[4] DS1× SB1A monoreassortant and a 100% protective efficacy against the Wa P[8] (FIG. 5C). Thus, the VP7-specific mAb #27 was able to inhibit RV-induced diarrhea in a VP7-serotype-specific manner, whereas VP7-specific mAbs #57 and VP5*-specific mAb #41 inhibited RV-induced diarrhea in a heterotypic manner.

RV vaccines, like several other orally administered vaccines (e.g., cholera, typhoid, and polio vaccines), have less efficacy in developing countries than in developed countries. Multiple factors likely account for this effect including higher frequency of microbial pathogen co-infections, elevated levels of breast milk IgA or transplacental IgG specific to the vaccine at the time of vaccination, malnutrition, micronutrient deficiencies, the force of infection in less developed versus developed countries and the distinct microbiome of the vaccine recipients in less developed countries. Furthermore, the substantial serotypic diversity of circulating wild-type human RV strains is likely an impediment to the development of broadly effective RV vaccines in especially in less developed countries where RV serotypic diversity is greatest.

It is generally the case that children develop substantial resistance to severe recurrent wild-type RV illness following one or two natural infections or following a single serotype (monotypic) vaccination series despite the serotypic diversity of RVs circulating in the environment and this situation holds true even in developing countries although the level of resistance is somewhat lower. The molecular basis of this broad resistance to multiple RV serotypes has remained an enigma for the past 30 years. Here we demonstrate that serotypic diversity of circulating wild-type RV strains is countered in humans by the common generation of broadly cross-neutralizing Ig molecules directed at either the RV VP7 surface glycoprotein or to the VP5* carboxy tryptic fragment of the surface attachment protein VP4.

Previous attempts to isolate and characterize human VP4- or VP7-specific B cells have been hampered by the fact that only very young children are readily susceptible to RV infection due to the existence of immunity in virtually everybody by the age of three or four. It is difficult to acquire acute-phase plasmablast-rich peripheral blood specimens for purely research purposes from this vulnerable, pediatric population. In addition, due to the intrinsic tendency of RV TLPs to uncoat during storage, labeling, and/or other experimental manipulations, it has been difficult to use authentic TLPs as capture antigens in flow cytometry-based assays to isolate RV-specific B cells directed at VP4 or VP7 surface antigens. Previous attempts by us and others to isolate VP4- or VP7-specific B cells using recombinant TLP-VLPs expressing GFP-VP2 resulted in the selection of numerous VP6-specific, rather than VP4- or VP7-specific, B cells.

Here we provide data demonstrating the resolution of these technical challenges. First, we took advantage of a naturally occurring, highly stable TLP-forming human RV strain CDC-9 in combination with optimized chemical labeling and single-cell sorting conditions. Second, we used a Cy5-labeled RV probe that had higher fluorescence intensity and less overlap with cellular autofluorescence than traditional GFP-labeled recombinant TLP-VLPs. This novel approach enabled identification of B cells with surface Ig specific for VP4 or VP7 with an excellent discovery rate of 53% (33/62) for RV-specific VP4- and VP7-directed B cells; only one VP6-specific mAb was identified (Table 1).

The frequency of TLP-binding VP4- and VP7-reactive B cells identified by FACS in the five donors analyzed in this study was comparable to the frequency of VP6-specific B cells identified by ELISPOT (FIG. 2B, C). This was not expected since VP6 is known to be the dominant target of the humoral immune response to RV. Of note, in addition to the fact that the two assays are not directly comparable, the specificity and sensitivity of the FACS assay is clearly impacted by non-specific binding due in part to the detection tag itself. Based on the discovery rate of VP4- and VP7-specific mAbs among expressed mAbs, we estimate the median frequency of true TLP-binding IgA⁺ ASCs among total IgA⁺ ASCs to be roughly 0.07%, which is roughly half the median frequency of VP6-specific IgA⁺ ASCs identified by ELISPOT (FIG. 2).

A barcode-based sequencing strategy was used to facilitate the efficient selection of natively paired, antigen-specific antibodies. The strategy can accurately identify clonal expansions, if present in the Ab repertoire, as a proxy for antigen-activated and expanded B cells. This approach has been shown to be highly effective in identifying clonally expanded and enriched antigen-specific plasmablasts with higher affinity and neutralizing capacity than singletons from the same patient, when applied to the analysis of peripheral antibody-secreting plasmablasts induced following recent vaccination, infection or other form of acute antigen exposure. In the present study, we use labeled antigen-specific bait to enrich for antigen-specificity in the steady state ASC repertoire from adult subjects who were unlikely to have an acute antigen-specific plasmablast response similar to those with recent vaccination or infection.

Although most of the RV-specific Abs we identified were present in clonal families, 33% were singletons (FIG. 3). We attempted to maximize our sampling size by isolating ASCs from the entire small bowel tissue resection and sorting and sequencing all identifiable TLP-binding B cells obtained from the five subjects. In this analysis, however, among the combinations of VH-VL gene segments that were shared across donors, none were present in mAbs with confirmed RV-binding specificity, while two VH-VL gene combinations were present in mAbs that did not specifically bind RV.

As might be expected given the adult age of our subjects and the ubiquitous nature of RV infection, the molecular features of the RV-specific intestinal repertoire revealed characteristics of antigen-mediated selection. The V gene segment mutation frequency, CDRH3 length, and the number of positively charged amino acids in CDRH3 (FIG. 4) were consistent with previous reports on intestinal IgA⁺ ASCs. RV-binding Abs that did not have neutralizing activity appeared to have fewer somatic mutations in their VH genes compared to genes encoding RV neutralizing Abs and Abs not characterized in terms of binding specificity in this study.

These studies were performed using human intestinal ASCs from RV-exposed adult bariatric surgery subjects for two primary reasons. First, the two licensed, orally administered RV vaccines in the USA do not reproducibly elicit a robust peripheral plasmablast response in adults, so we could not acquire blood samples with a reliable acute RV-specific plasmablast response from immunized adult volunteers. Second, previous studies established that RV-reactive ASCs are present in substantial numbers in the small intestine of healthy adults and non-immunodeficient adult mice months to years following RV exposure. The present findings confirm these observations since roughly 0.16% of all jejunal IgA secreting cells in the five adults in our study produced Abs directed at RV VP6; remarkably, in one donor 1.3% of all IgA⁺ ASCs secreted Abs to RV VP6 (FIG. 2).

The reasons underlying the long-term maintenance of high levels of RV-specific B cell immunity in the small intestine are unclear. In non-immunodeficient animals, RV infection is acute and RV is not thought to persist, although persistence of Group A RV genomes has been recently described in the adult bovine mesenteric lymph node. In humans, relatively frequent re-exposure to infectious RV might contribute to the persistence of high levels of RV-specific ASCs in the gut, but this explanation would not account for such persistence in the experimental mouse model where environmental re-exposure does not occur. Interestingly, a previous study showed that approximately 30% of intestinal IgA and IgG ASCs obtained from healthy donors were poly-reactive when tested against a panel of self-antigens, intestinal bacteria, and RV. The great majority of these Abs recognized RV VLPs expressing only VP2 and VP6. Only one of 137 IgA and two of 85 IgG plasmablast clones were exclusively specific for the RV VP2/VP6 DL-VLPs. Thus, the majority of intestinal plasmablasts that recognize RV VP6 appear to be poly-reactive. On the other hand, the VP4 and VP7 specific mAbs isolated in the current study appear to be highly RV specific.

In terms of their protein targets, the great majority (29/33) of the TLP-directed intestinal B cells isolated from the jejunal resections were VP4- rather than VP7-specific (Table 1), despite the fact that VP4 is stoichemetrically underrepresented on the virion surface compared to VP7. Consistent with this finding, RV VP4 has previously been reported in some, but not other studies, to be the dominant target of protective immunity in children following natural RV infection or vaccination. In adults experimentally inoculated with a virulent human RV challenge pool, VP4 was found to be the immune-dominant protein based on induction of neutralizing Abs. In another study, however, the immune response to VP7 epitopes showed a significant correlation with protection against infection and symptom development in adults challenged with a virulent wild-type serotype G1 RV strain. In the current study most intestinal mAbs that specifically bound to intact RV TLPs were VP4-specific (>87%) although the ratio of neutralizing VP7 to neutralizing VP4 mAbs was just 1:2 (Table 2). Specificity analyses indicated that murine mAbs to either VP4 or VP7 effectively bound to the Cy5-labeled TLPs (FIG. 1). The mean fluorescent intensity of TLP-Cy5 bound to VP4 P[8]-specific hybridomas was higher than that to VP7 G1-specific hybridomas, which may suggest a bias in the B cell selection assay that rendered isolation of VP4-directed cell surface Igs more efficient than isolation of those directed at VP7. Taking into account this possible caveat, our findings suggest that a far greater proportion of the B cell response to RV is directed at VP4 than VP7.

Of the nine TLP binding mAbs that actually neutralized RV, six targeted VP4 and three targeted VP7. Because fewer of the isolated mAbs were directed at VP7 than VP4, the fraction of mAbs with neutralizing activity was actually higher for VP7 (3/4) than for VP4 (6/29) (Tables 1 and 2). Not unexpectedly, most (79%) of the VP4-reactive mAbs were non-neutralizing in vitro and did not protect mice in passive transfer experiments. It was surprising, however, that not one of the 22 isolated mAbs directed at the VP8* fragment of VP4 possessed neutralizing capacity. This finding differs significantly from a large number of previously published studies of murine mAbs directed primarily at animal RVs in which VP8* was identified as a frequent target of neutralizing Abs. The majority of VP4- or VP7-specific mAbs examined prior to this study were murine in origin, were induced following parenteral immunization rather than enteric infection, and were identified by functional screening assays based on either neutralization of HAI assays, not binding assays. To the best of our knowledge, this is the first study of RV surface protein targeted mAbs in which screening was based solely on TLP binding rather than functional reactivity. Using this more unbiased isolation approach, neutralizing Abs would appear to represent only a limited subset of the immune repertoire generated to VP4 but perhaps a much larger proportional component (here, three fourths) of VP7-directed mAbs. The four VP7-specific mAbs identified here are too few to accurately predict what proportion of the human immune response to this protein can restrict RV replication, but this initial data suggests that most Abs that bind to the trimeric form of VP7 found on the RV surface are likely to inhibit viral replication, presumably by impeding viral uncoating.

The majority of VP4-reactive mAbs and all 22 of the VP8*-specific mAbs we isolated were inactive in traditional neutralization or passive neutralizing, and four of the five VP5*-specific neutralizing mAbs were broadly heterotypic both in vitro and in vivo (Table 2, FIG. 5). Previously, RV neutralizing epitopes have been mapped to both VP5* and VP8* antigenic domains on VP4 using mAbs derived from mice parenterally immunized with either animal or human RVs. The VP8*-directed murine mAbs have generally been type-specific, in keeping with the relatively high degree of sequence divergence in this region of the molecule. VP5*-directed murine mAbs have demonstrated more cross-reactive serologic specificity. The cross-reactivity of anti-VP5* Abs is consistent with the relative sequence conservation of this region and functional constraints on this portion of the molecule due to its role in membrane passage during cell entry. It is interesting to note that, like VP7, we have failed to identify any VP5* directed Abs that lacked the ability to restrict RV replication suggesting that most of the VP5* antigenic surface that is exposed on TLPs likely plays an important role in mediating viral infection.

The Ab-antigen co-evolution of heterotypic immunity to RV may have occurred in a manner similar to what is observed for broadly neutralizing human mAbs against the influenza membrane proximal HA stalk domain and HIV-1 envelope glycoprotein, both of which target receptor binding sites and membrane fusion machinery. The precise atomic binding sites of the broadly heterotypic human VP5* mAbs described here as well as their mechanism of neutralization await additional studies; however, such Abs are unlikely to function by inhibiting viral binding but might be involved in restricting cell entry. Prior studies using experimentally induced murine mAbs identified amino acid regions 248 to 474 as critical sites for the binding of heterotypic VP5*-directed heterotypic mAbs. Direct evidence of the involvement of this epitope in mediating protection in children also has been demonstrated. The Abs we examined were derived from adults who have likely undergone multiple RV exposures. Whether the high proportion of highly heterotypic VP5*-directed Abs is established during initial RV infection or vaccination or requires time and multiple exposures to develop can be determined by a similar analysis of very young children undergoing primary infection or vaccination

Of note, truncated VP8* subunit protein vaccine candidates containing most of the neutralizing epitopes expressed on VP8* have recently been shown to elicit RV-neutralizing Ab responses in animal models and to boost neutralizing Ab titers in RV-experienced adults when administered parenterally. It is surprising that none of the 22 individual anti-VP8* Abs isolated in our study had neutralizing activity in vitro in traditional cell culture assays, neutralization assays using human intestine derived organoids, or in passive protection challenge experiments in suckling mice. Whether these negative results represent a sampling error due to the limited number of adults studied, the restriction of our study population to obese adults, an unknown bias in our B cell selection strategy, or whether VP8*-neutralizing epitopes are occluded or differentially presented in the intestinal milieu of people, will be determined as additional human mAbs to RV are isolated and characterized and as the neutralizing immune response to recombinant VP8*-based vaccines in immune-naïve children is examined. However, the negative results seen here provide some degree of caution regarding the potential for VP8* to function as an effective third generation human RV vaccine candidate.

The VP8* fragments of VP4 of the major human RV serotypes interact with several distinct human histo-blood group antigens (HBGA), expressed on mucosal epithelial and other cell types. Genetic and developmental variation in HBGA expression may result in variable susceptibility to infection with different RV strains. P[8] and P[4] strains share reactivity with the common Lewis b (Le^{b}) and H type 1 antigens, whereas P[6] strains bind the H type 1 antigen only. Most VP8*-specific mAbs identified in this work bound to VP4 from both P[8] and P[4] strains (18/22). It is not clear, at present, why the multiple Abs to VP8* failed to neutralize given the importance of this protein in the initial cell binding functions of the virus. Presumably the mAbs we isolated bind to VP8* regions that are not directly involved in cell surface binding. Structural analysis and blocking experiments with various glyco-array libraries will be needed to better understand the molecular basis of this unexpected finding.

In summary, our findings provide a highly plausible molecular explanation to the long-standing and fundamental question regarding how heterotypic immunity to RV illness is mediated after natural infection or monotypic vaccination despite the very substantial serotypic diversity of circulating human RV strains in the environment. In addition, these studies suggest that recombinant vaccines containing or capable of expressing VP4 or, more specifically VP5*, will be the most promising approaches to develop third generation, non-replicating RV vaccine candidates to enhance immunity in less developed countries where the efficacy of oral immunization is not optimal. Ongoing structural studies to map the neutralizing B cell epitopes and the atomic structures recognized by the functional VP5* and VP7 heterotypic mAbs will aid in the design of such improved, next-generation RV vaccines that could better address the burden of continuing RV disease in developing countries. Abs from additional subjects must be examined to confirm that VP4 is truly the dominant target of the immune response in adults. It will also be necessary to determine at atomic resolution the regions of VP5* that are the targets of heterotypic neutralizing Abs. In addition, follow-up studies are desirable to define the extent to which the findings based on adult intestinally derived ASCs presented here accurately recapitulate the B cell responses and specificities induced in young children in developed and developing countries following primary and secondary infection or vaccination.

### Methods

Human subjects. Proximal jejunum tissue resections were obtained from adults undergoing bariatric surgery at the Stanford University Hospital in accordance with Stanford University IRB protocols (IRB Protocol 13813). Exclusion criteria included chronic viral infections or acute gastroenteritis at the time of surgery.

Isolation of B cells from jejunum tissue and peripheral blood. Jejunum tissue resections were processed within 2 h of surgery. Viable mononuclear cells representative of the lymphoid population present in the gastrointestinal mucosa were isolated as previously described [44]. Briefly, tissue fragments were digested for 1 h at 37° C. with 0.26 Wünsch units/ml Liberase TL (Roche). Intestinal B cells were enriched using EasySep Human B cell Enrichment Kits without CD43 Depletion (Stemcell Technologies) according to the manufacturer's instructions. Isolated B cells were incubated at 37° C. in 5% CO₂ for 2 h prior to staining.

RV strains, propagation and preparation of TLPs, DLPs, and VLPs. RVs (Wa, DS1, RRV, ST3, OSU, NCDV, CDC-9, D×RRV, DS1×RRV, ST3×RRV, UK, 69M, 116E, #321, W161, L26) were grown in MA-104 cells (ATCC) in the presence of trypsin as described [88]. TLPs were purified from MA-104 cell lysates by genetron extraction, centrifugation through a sucrose cushion, and cesium chloride (CsCl) density gradient centrifugation as described [89, 90]. Purified TLPs were dialyzed to remove residual CsCl. DLPs were prepared by treating TLPs with 5 mM EDTA for 20 min at 37° C. VP2-eGFP/VP6 particles were prepared as previously described.

TLP preparation and labeling. TLPs (CDC-9) were labeled with Cy5 as described [92] with some modifications. Varying molar ratios of Cy5 to TLP were tested to determine the TLP-Cy5 conjugate that yielded the highest signal to noise ratio in FACS staining with VP4- and VP7-specific hybridoma cells (data not shown). Briefly, TLPs (100 μg) were washed twice with 10 mM Hepes, pH 8.2, 5 mM CaCl₂, 140 mM NaCl and labeled at 4:1 molar ratio of Cy5 mono-reactive dye (GE Healthcare) to TLP at room temperature for 1 h with gentle agitation. The labeling reaction was stopped with the addition of Tris-HCl, pH 8.8, to a final concentration of 50 mM. Labeled viruses were separated from unbound Cy5 by dialysis using Amicon Ultra Centrifugal filter unit (Millipore). The integrity of TLP-Cy5 compared to unlabeled TLP was determined by electron microscopy as described.

Flow cytometry. Murine hybridomas VP6 (1e11), VP4 P[8] (1a10) or P[3] (7a12), VP7 G1 (5e8) or G3 (159)) or enriched intestinal B cells were stained with TLP-Cy5 (2 μg) for 45 min on ice as previously described with modifications. The concentration of TLP-Cy5 required per staining reaction was determined in titration experiments on VP4-, VP7-, and VP6-specific hybridomas. Intestinal B cells were stained with LIVE/DEAD Fixable Acqua Dead Cell Stain Kit (Life Technologies) and a fluorescently-tagged Ab panel consisting of anti-CD3-PE Cy7 (clone: SKY), anti-CD14 PE Cy7 (clone: M5E2), anti-CD16-PE Cy7 (clone: 3G8), anti-CD20-APC H7 (clone: 2H7), anti-CD27-PE (clone: MT271), and anti-CD38 PerCP-Cy5.5 (clone: HIT2) all from Becton Dickinson and anti-IgA FITC (clone: IS11-8E10, Miltenyi Biotec). IgA⁺ ASCs were identified by gating on live, single cells and CD3/14/16⁻ CD20^{lo/-} CD27^{hi} CD38^{hi} IgA⁺ surface expression. IgA⁺ ASCs were bulk sorted using the Becton Dickinson FACS Aria III. The bulk-sorted population was then single-cell sorted into a 96-well PCR plate containing 10 mM Tris-HCl, pH 7.6, 2 mM dNTPs (New England Biolabs), 5 μM oligo (dT) and 1 unit/μl Ribolock (Thermo Scientific). At least 200,000 events were acquired per sample. Data were analyzed using Cytobank [94].

ELISPOT. The frequencies of intestinal IgA⁺ ASCs and VP6⁺ IgA⁺ ASCs were determined by ELISPOT as described.

Barcode-based sequencing of paired IGH and IGL genes. Reverse transcription (RT) and PCR with well-ID and plate-ID oligonucleotide barcode adaptors was performed as described [45]. Briefly, 6 mM MgCl₂ with Ribolock, Superscript III (Life Technologies), and 1 μM of the appropriate well-ID oligonucleotide barcode were added to the sorted ASCs in individual wells of 96-well plates and RT was performed at 42° C. for 120 min. RT products from each plate were pooled. PCR1 was performed with forward (FW) primers containing a 5′ plate-ID barcode oligonucleotide and a 454 titanium adaptor, and with reverse primers specific for mRNAs encoding the Ig alpha, kappa, and lambda chains. PCR2 was performed using FW primers with a 5′ 454 titanium adaptor and reverse GSP with a 3′ plate-ID barcode oligonucleotide and a 454 titanium adaptor. Amplified DNAs were pooled, purified with Ampure XP beads (Beckman Coulter) and sent to Roche for 454 sequencing. Compound barcode assignment, assembly of sequences, V(D)J and clonal assignment and clustering of sequences were performed essentially as described. Original nucleotide sequences were submitted to GenBank. IMGT HighV-Quest data were read into R, and B cells with shared HC VJ and LC VJ gene segments were clustered. Within these groups, CDR3 AA sequences were compared using the stringdist package to calculate Levenshtein distance. Clonal families were defined as sharing HC and LC VJ genes and having a CDR3 amino acid Levenshtein distance of ≤3 for both. Clonal families were numbered and counted in R prior to statistical analysis with GraphPad Prism. For analysis of combinatorial VH/VL gene usage across donors, the frequency of each IGH VJ and IGK/L VJ gene usage combination was calculated for each individual, and the values were normalized to account for differences in sequencing depths between the subjects.

Cloning and expression of recombinant Abs. Ab cloning and expression were performed as described with modification. V(D)J gene regions from Ig alpha and gamma heavy chain and from kappa or lambda light chains were synthesized (Integrated DNA Technologies) and initially inserted into pFUSE-CHIg1-hG1 (IgG1) or pFUSE-CHIg-hAI (IgA1) and pFUSE2-CLIg-hK (IgK) or pFUSE2-CLIg-hL2 (IgL) expression vectors (InvivoGen), respectively, using the SRI Cold Fusion Cloning reaction kit. Plasmids encoding heavy and light chain V(D)J inserts were co-transfected into Expi293T cells (Life Technologies). Subsequently, for consistent expression and to improve secretion of the mAbs, V(D)J sequences were inserted into expression vectors containing interleukin-2 leader sequence (pFUSEss-CHIg-hG1 (IgG1), pFUSE2ss-CLIg-hK (IgK), pFUSE2ss-CLIg-hL2 (IgL)) (InvivoGen). Supernatants were harvested after 5 days and assayed for IgG or IgA expression.

ELISAs. The quantity of total IgG or IgA was assessed in transfection supernatants using the Human IgG or IgA ELISA kit (Zeptomatrix) and by fitting the standard curve to the 4 parameter logistic nonlinear regression model using Softmax Pro 6.5 (Molecular Devices). To determine binding reactivity to RV proteins, immunoplates (Thermo Fisher) were coated with TLPs (CDC-9), VLPs VP2-eGFP/VP6, bacterially expressed VP8* conjugated to tetanus toxoid [84] (a gift from PATH, Seattle, Wash.), or VP5* (1 μg/ml) overnight at 4° C. VP5* was produced via in vitro transcription and translation as described. Plates were washed with PBS containing 0.05% Tween-20 (Sigma-Aldrich) and incubated with serially diluted transfection supernatants for 2 h at 37° C. Plates were washed as described and incubated with goat anti-human IgG or IgA horse radish peroxidase (HRP) from KPL for 1 h at 37° C. Following washing, TMB substrate was added followed by addition of 2% H₂SO₄ to stop the reaction. Optical density was read at 450 nm using an ELx800 microplate reader (BIO-TEK Instruments). All samples were run in duplicate. 1e11 (VP6), Yo-2C2 (VP4), and KU4 (VP7) mAbs were used as controls.

Immunostaining. Immunostaining was performed as previously described [50]. Recombinant BVs expressing VP7 (G1) or VP4 (Ku, DS-1, 1076) were used to infect Sf9 cells at a multiplicity of infection of 0.1. Infected Sf9 cells were fixed with 10% formalin (Sigma) for 30 min at room temperature, and permeabilized with 1% Triton X-100 (Sigma) in TNC (10 mM Tris, 100 mM NaCl, 1 mM CaCl₂, pH 7.4) for 2 min at room temperature as previously described [50]. mAbs were serially diluted and incubated for 1 h at 37° C. mAbs that bound to specific BV-infected Sf9 cells were detected with HRP-labeled goat anti-human IgG or IgA (KPL), followed by incubation with 3-amino-9-ethyl-carbazole (AEC) (Vector Laboratories). The endpoint immunostaining concentration was assigned as the highest dilution at which cell staining could be detected using an inverted microscope. To determine the binding reactivity of recombinant mAbs to specific RV strains, MA104 cells were infected with specific RVs strains as indicated. Cells were fixed and permeabilized. mAbs were used to stain intracellular RVs and binding reactivity was detected using HRP-conjugated goat anti-human IgG or IgA as described. All samples were run in duplicate and each assay was repeated twice.

Immunoprecipitation. MA104 cells were infected with human RV Wa strain at multiplicity of infection of 3. At 16 h post infection, total RNA was isolated using the RNeasy Mini Kit (Qiagen) according to manufacturer's instructions. cDNA was prepared from the isolated RNA using High Capacity cDNA Reverse Transcription Kit (Applied Biosystems). VP4, VP5*, and VP8* coding sequences were amplified using Phusion polymerase (New England Biolabs) and the primers listed in Table 4. Amplified sequences were cloned into pCMV6-XL6 vector (Origene) containing SP6 promoter using KpnI and HindIII restriction sites.

VP4, VP5*, and VP8* proteins were translated in vitro using TNT®Quick Coupled Transcription/Translation Systems (Promega) with rabbit reticulocyte lysate and SP6 polymerase. The translated proteins or whole virus particles were mixed with human anti-RV mAbs and incubated overnight at 4° C. with continuous mixing. The protein-mAb complexes were then incubated for 1 hour at room temperature with PureProteome Protein A/G magnetic beads (Thermo Scientific) and precipitated using a magnetic field. The immune-complexes were resolved in denaturing SDS-PAGE and immunoblotted to PVDF membrane. The membranes were probed with anti-VP5* IgG (clone: HS-2). Immunoprecipitated proteins were visualized using ECL Plus western blotting substrate (Thermo Scientific).

Virus neutralization assays. Virus neutralization assays were performed as described [96]. Briefly, recombinant mAbs (5 μg/ml) were serially diluted, and the dilutions were mixed with the following RV strains: Wa, CDC-9, D×RRV, DS1, DS1×RRV, RRV, ST3, ST3×RRV, OSU, NCDV, UK, W161, L26, 69M, 116E and 321 for 1 h at 37° C. The mAb-virus mixture was transferred to MA-104 cell monolayers in a 96-well plate and incubated for 1 h at 37° C. in 5% CO₂. The antibody and virus mixture was removed, and cells were washed twice and incubated overnight at 37° C. with 100 μl of M199 media without serum or trypsin. The cells were fixed with 10% formalin for 30 min and permeabilized with 1% Triton X-100 for 2 min. After washing, polyclonal rabbit anti-RV IgG was added to the plate for 2 h at 37° C. The plate was washed and HRP-conjugated goat anti-rabbit IgG (γ chain specific) (Sigma-Aldrich) was added. After 1 h incubation at 37° C., a color reaction was detected with the AEC substrate. The neutralization activity was defined as the highest dilution at which virus-positive foci were reduced by at least 50% compared to the controls untreated with mAb and expressed as minimum neutralization concentration (ng/ml). All samples were run in duplicate, and each assay was repeated twice.

Organoid RV infection. Duodenal derived primary human intestinal organoids (kindly provided by Calvin Kuo, Stanford University) were cultured and infected with RV as previously described with minor modifications [97]. Briefly, 3D cultures of organoids in Matrigel (Corning) were maintained in growth media consisting of DMEM-F-12 supplemented with growth factors including epidermal growth factor (EGF) (Invitrogen), Noggin (Peprotech), R-spondin (Peprotech), Wnt3A (R&D Systems), nicotinamide (Sigma), gastrin I (Sigma), SB202190 (Sigma), B27 supplement (Invitrogen), N2 supplement (Invitrogen), and acetylcysteine (Sigma). Two days prior to the human mAb neutralization assays, the organoid cultures were switched to differentiation media comprised of growth media without Wnt3A, BS202190, or nicotinamide and with a 50% reduction of Noggin and R-spondin. Human mAb #41 (5 μg/ml) or mixtures of VP8*-specific mAbs (mAb #4, #9, #16, #18, and #20 at 5 μg/ml) were incubated with Wa (10⁵ PFU) for 1 h at 37° C. The organoids were treated with TrypLE (Gibco) and co-incubated with Wa-mAb mixtures for 1 h at 37° C. After incubation, new Matrigel was added to the Wa-mAb mixture, and the infected organoids were cultured in differentiation media for a total of 24 h. The organoids were then permeabilized in buffer containing 3% BSA, 1% saponin and 1% triton X-100 and then stained with anti-VP6 FITC (clone 1e11), anti-phalloidin Texas Red, and 4′,6-diamidino-2-pheylindole (DAPI) (both from Invitrogen). RV infection in the organoids was quantified using a Keyence BZ-X710 all-in-one fluorescence microscope.

Mouse passive challenge studies. 129/Sv mice were originally purchased from Taconic Biosciences. Sucking mice were bred in the VA Palo Alto Health Care System Veterinary Medical Unit. RVs were incubated with RV neutralizing human mAbs (5 μg/ml) for 1 h at 37° C., and the RV-mAb mixture was then used to orally gavage 5 day old 129/Sv suckling mice. Human anti-VP7 mAbs (mAb #27 and mAb #57) were mixed with RRV or D×RRV and human anti-VP4 mAb (mAb #41) was mixed with Wa or DS1× SB1A. Six to 11 mice were included per group. The RV dose for each inoculum was 10⁶ PFU. Mice were monitored for 4 days for diarrheal disease. All experiments were conducted in accordance with Stanford University and the VA Palo Alto Health Care System guidelines. mAb protective efficacy was calculated as: diarrhea rate of RV-infected control mice minus diarrhea rate of RV infected and mAb treated mice divided by the diarrhea rate of RV-infected control mice.

Statistics. Statistical analyses were performed using GraphPad Prism (version 6.0b). One way ANOVA was used to compare differences between multiple groups. The unpaired t-test was used to compare differences between two groups. P values <0.05 were considered significant.

Study Approval. This study was approved by the institutional review board of Stanford University. Written informed consent was obtained from all patients prior to inclusion in this study. Sequences.

Sequence identifiers of MAb nucleotide and protein sequences are provided below. The mAb ID corresponds to the identification numbers used in, for example, Tables 1-3. As indicated in the Tables, each of monoclonal antibodies 2, 27, 30, 33, 41, 46, 47, 49 and 57 show neutralizing activity in vitro. Monoclonal antibodies 27, 41 and 57 have demonstrated in vivo neutralization activity. Monoclonal antibodies 2, 30, 33, 41 and 49 bind to VP5*; and 2, 30, 41 and 49 are heterotypic.

| | | |
|---|---|---|
| mAb ID: 2 | Heavy Chain coding sequence | SEQ ID NO: 1 |
| | Light chain coding sequence | SEQ ID NO: 2 |
| mAb ID: 27 | Heavy Chain coding sequence | SEQ ID NO: 3 |
| | Light chain coding sequence | SEQ ID NO: 4 |
| mAb ID: 30 | Heavy Chain coding sequence | SEQ ID NO: 5 |
| | Light chain coding sequence | SEQ ID NO: 6 |
| mAb ID: 33 | Heavy Chain coding sequence | SEQ ID NO: 7 |
| | Light chain coding sequence | SEQ ID NO: 8 |
| mAb ID: 41 | Heavy Chain coding sequence | SEQ ID NO: 9 |
| | Light chain coding sequence | SEQ ID NO: 10 |
| mAb ID: 46 | Heavy Chain coding sequence | SEQ ID NO: 11 |
| | Light chain coding sequence | SEQ ID NO: 12 |
| mAb ID: 47 | Heavy Chain coding sequence | SEQ ID NO: 13 |
| | Light chain coding sequence | SEQ ID NO: 14 |
| mAb ID: 49 | Heavy Chain coding sequence | SEQ ID NO: 15 |
| | Light chain coding sequence | SEQ ID NO: 16 |
| mAb ID: 57 | Heavy Chain coding sequence | SEQ ID NO: 17 |
| | Light chain coding sequence | SEQ ID NO: 18 |
| mAb ID: 2 | Heavy Chain protein sequence | SEQ ID NO: 19 |
| | Light chain protein sequence | SEQ ID NO: 20 |
| mAb ID: 27 | Heavy Chain protein sequence | SEQ ID NO: 21 |
| | Light chain protein sequence | SEQ ID NO: 22 |
| mAb ID: 30 | Heavy Chain protein sequence | SEQ ID NO: 23 |
| | Light chain protein sequence | SEQ ID NO: 24 |
| mAb ID: 33 | Heavy Chain protein sequence | SEQ ID NO: 25 |
| | Light chain protein sequence | SEQ ID NO: 26 |
| mAb ID: 41 | Heavy Chain protein sequence | SEQ ID NO: 27 |
| | Light chain protein sequence | SEQ ID NO: 28 |
| mAb ID: 46 | Heavy Chain protein sequence | SEQ ID NO: 29 |
| | Light chain protein sequence | SEQ ID NO: 30 |
| mAb ID: 47 | Heavy Chain protein sequence | SEQ ID NO: 31 |
| | Light chain protein sequence | SEQ ID NO: 32 |
| mAb ID: 49 | Heavy Chain protein sequence | SEQ ID NO: 33 |
| | Light chain protein sequence | SEQ ID NO: 34 |
| mAb ID: 57 | Heavy Chain protein sequence | SEQ ID NO: 35 |
| | Light chain protein sequence | SEQ ID NO: 36 |

Exemplary protein sequences identified by the methods described herein are provided below. The underlining indicates exemplary CDR sequences, although those of skill in the art will recognize that various algorithms can be used for the identification of CDR sequences, and there can be minor variations as a result.

mAb ID: 2 comprises the heavy chain variable region
(SEQ ID NO: 19)
IGHEVQLVESGGGLVKPGGSLRLSCKASGLIVSDAWMSWVRQSPGKGLEWVGRIKSEINGGTI
DYAAPVKGRFTILRDDSKNTLYLQINSLKTEDTAVYYCTTRLLFSPWGQGTLVTVSS,
and
the light chain variable region
(SEQ ID NO: 20)
QPVLTQPPSSSASPGESARLTCTLPSDINVAYYNIYWYQQKPGSPPRYLLYYYSDSDQGQGS
GVPSRFSGSKDASANTGILFISGLQSEDEADYYCMIWTSNASMFGGGTKLTVL
mAb ID: 27 comprises the heavy chain variable region
(SEQ ID NO: 21)
IGHQVQLQESGPGLVKPLETLSLTCAVSGVSINSYYWSWIRQPPGKGLEWIGNVFYSGSTKYN
PSLESRVAMTVDSSRNQVSLRLNSVTAADTAVYYCAREGVGYGYNNYGGNWFDPWGQGTL
VTVSS
and
the light chain variable region
(SEQ ID NO: 22)
EVVLTQSPGTLSLSPGERVTLSCRASQSVTSSNLAWYQQKPGQTPRLLISGASSRATGIPDRF
SGSGSGTDFTLTISRLEPEDFAVYYCQQYANSPVTFGGGTKLEIK
mAb ID: 30 comprises the heavy chain variable region
(SEQ ID NO: 23)
IGHQVQLVQSGAEVKKPGASVTVSCKASGYAFTSFYLHVVVRQAPGQGLEWMGIINPSDGRTR
YAQKFQGRVTMTSDTSTNTVYVELSSLRSEDTAIYYCARGAIGNYNAREALDVWGRGTTVTVS
S
and
the light chain variable region
(SEQ ID NO: 24)
EIVMTQSPATLSVSPGESATLSCRASQSINSNLAWYQQKPGQAPRLLIFSASSRATGIPARFSG
SGSGTEFTLTISSLQSDDFAVYYCQQYNIWPPEHTFGQGTRLQIK
mAb ID: 33 comprises the heavy chain variable region
(SEQ ID NO: 25)
IGHDVQLVESGGGLVQPGGPSRLSCSASRFTFSNYAMYWVRQAPGKGLEYVSSISSDGGSTY
YAESVKGRFTISRDNSKNTLYLQMRSLRAEDAAVYYCVTDVLRLPYSTGWSPGDFIYWGQGT
LVTVSS
and
the light chain variable region
(SEQ ID NO: 26)
DIQMTQSPSILYASVGDRVTITCRASQSVSSWLAWYQQKPGKVPKLLIYQASTLENGVPSRFS
GSGSGTEFILTISSLQPDDFATYYCQHYNVLwTFGQGTKVEI
mAb ID: 41 comprises the heavy chain variable region
(SEQ ID NO: 27)
IGHEVQLVESGGGPVQPGGSLKLSCAASGFTFSNYEMYWVRQAPGKGLEWVSYISTSPAITY
YADSVRGRFTISRDNAKSSLYLHMNSLRAEDTAVYYCATISHQQFSSGWNAWFDPWGQGTLV
TVSS
and
the light chain variable region
(SEQ ID NO: 28)
NFMLTQPHSVSESPGKTVTISCTGSSGSIASNYVQWYRQRPGSAPTTVIYENYQRPSGVPARF
SGSIDRSSNSASLTISGLQTDDEADYYCQSYDNNNLWVFGGGTKLTVL
mAb ID: 46 comprises the heavy chain variable region
(SEQ ID NO: 29)
QVQLQESGPGLVKPSETLSLTCTVSGGSINSYYWSWIRQSPGKGLEWIGYVFYSGITKYNPSL
QSRVTISLDMGKNQFSLKLTSVNAADAAVYYCARNFPSYTPDWFFDLWGRGTLVTVSS
and
the light chain variable region
(SEQ ID NO: 30)
EIVLTQSPGTLSLSPGERATLSCRASQSVSSDNLAWYQQKPGQPPRLLIYGASHRATGIPDRF
SGSGSGTDFTLTISRLEPEDFAVYHCQQYGSSPLTFGGGTKVEIK
mAb ID: 47 comprises the heavy chain variable region
(SEQ ID NO: 31)
QVQLQESGPGLVKPSETLSLTCSVSGGSISVYYWNWIRQSPGKGLEWIASMYYTGITNYNPSL
KSRVTMSVDMSKNQFSLKLSSVTAADTAVYYCARTMGIDQNNRGWPPAGYYFGMDVVVGQG
TTVTVSS
and
the light chain variable region
(SEQ ID NO: 32)
DIVMTQSPLSLPVTPGEPASISCRSSQSLLHSNGNNYLDWYLQKPGQSPQLLIYLGSNRASGV
PDRFSGSGSGTDFTLKISRVEAEDVGIYYCMQALEASLTFGGGTKVEIK
mAb ID: 49 comprises the heavy chain variable region
(SEQ ID NO: 33)
DVQLVESGGGLVQPGGSVRLSCSASRFTFSNYAMYWVRQAPGKGLEYVSSISSDGGSTYYA
ESVKGRFTISRDNSKNTLYLQMRSLRAEDAAVYYCVTDVLRLPYSTGWSPGDFIYWGQGTLVT
VSS
and
the light chain variable region
(SEQ ID NO: 34)
DIQMTQSPSILYASVGDRVTITCRASQSVSSWLAWYQQKPGKVPKLLIYQASTLENGVPSRFS
GSGSGTEFILTISSLQPDDFATYYCQHYNVLVVTFGQGTKVEIK
mAb ID: 57 comprises the heavy chain variable region
(SEQ ID NO: 35)
QVQLVESGGGVVQSGRSLRLSCAASGFTFRSYAMHWVRQAPGKGLEWVADLSLDGSHKYA
DSVRGRFTISSDSSKNTVYLQMNSLRTEDTAIYYCARAAGIMVAGTFLTEFYFDYWGQGTLVT
VSS
and
the light chain variable region
(SEQ ID NO: 36)
QSVLTQPPSVSGAPGQRVTISCTGSSSNIGAGYDVHWYQQLPGTAPKLLIYGNIKRPSGVPDR
FSGSKSGTSASLAITGLQTEDEADYYCQSYDSSLSAYYVFGTGTRVTVL.

TABLE 1
Summary of the protein specificity of rotavirus-specific
human recombinant monoclonal antibodies

| | | | Minimum | | | |
|---|---|---|---|---|---|---|
| | | | concentration | G serotype/ | | Clonal |
| | | | for TLP | P genotype | In vitro | family |
| Mab. | Viral protein | | binding | binding | neutralization | or |
| No. | specificity | | (ng/ml) | activity | activity | singleton |
| 2 | VP4 | VP5* | 5.00 | P[4], P[8] | Y | CF |
| 30 | | VP5* | 5.00 | P[1], P[4], P[6], P[7], P[8] | Y | CF |
| 33 | | VP5* | 0.50 | P[1], P[3], P[4], | Y | CF |
| | | | | P[6], P[7], P[8] | | |
| 41 | | VP5* | 0.01 | P[1], P[3], P[4], | Y | CF |
| | | | | P[6], P[7], P[8] | | |
| 49 | | VP5* | 5.00 | P[1], P[3], P[4], | Y | CF |
| | | | | P[6], P[7], P[8] | | |
| 4 | | VP8* | 0.50 | P[4], P[8] | N | CF |
| 6^{A} | | VP8* | 5.00 | P[4], P[8] | N | CF |
| 8 | | VP8* | 0.05 | P[4], P[8] | N | CF |
| 9 | | VP8* | 0.01 | P[4], P[8] | N | CF |
| 11 | | VP8* | 0.01 | P[4], P[8] | N | CF |
| 12 | | VP8* | 0.05 | P[4], P[8] | N | CF |
| 13 | | VP8* | 5.00 | P[8] | N | CF |
| 14 | | VP8* | 0.50 | P[4], P[8] | N | CF |
| 15 | | VP8* | 0.50 | P[4], P[8] | N | S |
| 16 | | VP8* | 0.01 | P[4], P[8] | N | CF |
| 18 | | VP8* | 0.05 | P[4], P[6], P[8] | N | CF |
| 19 | | VP8* | 0.05 | P[4], P[8] | N | CF |
| 20 | | VP8* | 0.01 | P[4], P[8] | N | S |
| 21 | | VP8* | 0.50 | P[4], P[8] | N | S |
| 23 | | VP8* | 50.00 | P[4], P[8] | N | S |
| 29 | | VP8* | 0.05 | P[8] | N | S |
| 31 | | VP8* | 0.50 | P[4], P[8] | N | CF |
| 35 | | VP8* | 0.50 | P[1], P[4], P[7], P[8] | N | S |
| 43 | | VP8* | 0.50 | P[8] | N | S |
| 44 | | VP8* | 0.50 | P[4], P[8] | N | CF |
| 55 | | VP8* | 500.00 | P[4], P[6], P[8] | N | CF |
| 60 | | VP8* | 0.50 | P[1], P[3], P[6], P[8] | N | CF |
| 47 | | ND | 0.50 | P[4], P[8] | Y | S |
| 62 | | ND | 5.00 | P[4], P[8] | N | CF |
| 22 | VP7 | | 5.00 | G1 | N | S |
| 27 | | | 0.01 | G1, G5, G9 | Y | CF |
| 46 | | | 0.50 | G1 | Y | S |
| 57 | | | 0.50 | G1, G2, G3, G4, G5 | Y | S |
| 10^{A} | VP6 | | 0.5^{B} | G1, G2, G3, G4, G5, G6 | N | CF |

^{A}IgA. All other mABs are IgG.
^{B}reacts to TLP and DLP. All other mAbs react only to TLP.
CF, Clonal family
S, Singleton
ND, VP5* or VP8* specifically not determined

TABLE 2
Neutralization titers of rotavirus-specific human monoclonal antibodies against distinct RV serotypes

| | RV | | RV strain neutralized (G, [P])^{A} | | | | | | | | |
|---|---|---|---|---|---|---|---|---|---|---|---|
| Mab | protein | | Wa | DS1 | RRV | ST3 | OSU | NCDV | WI61 | L26 | Neutralizing |
| No. | specificity | | G1P[8] | G2P[4] | G3P[3] | G4P[6] | G5P[7] | G6P[1] | G9P[8] | G12P[4] | activity |
| 2 | VP4 | VP5* | — | 4.9 | — | — | — | — | 4.9 | 625.0 | Heterotypic |
| 30 | | VP5* | 0.6 | 1.2 | — | 9.8 | 78.1 | 312.5 | 1.2 | — | Heterotypic |
| 33 | | VP5* | 2.4 | — | 78.1 | 78.1 | 312.5 | 156.3 | — | — | Homotypic |
| 41 | | VP5* | 0.6 | 39.1 | 9.8 | 4.9 | 78.1 | 19.5 | 2.4 | 4.9 | Heterotypic |
| 49 | | VP5* | 39.1 | — | 19.5 | — | 156.3 | 39.1 | — | — | Heterotypic |
| 47 | | ND | 4.9^{B} | 4.9 | — | — | — | — | 78.1 | — | Heterotypic |
| 27 | VP7 | | 0.3 | — | — | — | 312.5 | — | 156.3 | — | Homotypic |
| 46 | | | 2.4 | — | — | — | — | — | — | — | Homotypic |
| 57 | | | 2.4 | 4.9 | 1.2 | — | 19.5 | — | — | — | Heterotypic |

^{A}minimum neutralizing concentration (ng/ml)
^{B}neutralized CDC-9 (G1P[8]) but not Wa
— no neutralizing activity
ND,VP5*, VP8* specifically not determined

TABLE 3
Molecular characteristics of RV-neutralizing human mococclonal antibodies

| Mab | RV protein | | IGH | | | IGL | | | CDRH3 amino | |
|---|---|---|---|---|---|---|---|---|---|---|
| No. | specificity | | VH | DH | JH | L or K | VL | JL | acid sequence | neutralizing reactivity |
| 2 | VP4 | VP5* | 3-15*01 | 2-21*02 | 5*02 | L | 5-37*01 | 3*02 | TTRLLFSP | Heterotypic, P[4], P[8] |
| 30 | | VP5* | 1-46*01 | 2-1*01 | 6*02 | K | 3-15*01 | J2*01 | ARGAIGNYNAREALDV | Heterotypic, P[4], |
| | | | | | | | | | | P[6], P[8] |
| 33 | | VP5* | 3-64D*06 | 6-19*01 | 4*02 | K | 1-5*03 | 1*01 | VTDVLRLPYSTGWSPGDFIY | Heterotypic, P[8] |
| 41 | | VP5* | 3-48*03 | 6-19*01 | 5*02 | L | 6-57*01 | 3*02 | ATISHQQFSSGWNAWFDP | Heterotypic, P[3], |
| | | | | | | | | | | P[4], P[6], P[8] |
| 49 | | VP5* | 3-64D*06 | 6-19*01 | 4*02 | K | 1-5*03 | 1*01 | VTDVLRLPYSTGWSPGDFIY | Heterotypic, P[1], |
| | | | | | | | | | | P[3], P[8]^{A} |
| 47 | | ND | 4-59*01 | 6-19*01 | J6*02 | K | 2-28*01 | 4*01 | ARTMGIDQNNRGWPPAGYYF | Heterotypic, P[4], P[8] |
| | | | | | | | | | GMDV | |
| 27 | VP7 | | 4-59*01 | 5-24*01 | 5*02 | K | 3-20*01 | 4*01 | AREGVGYGYNNYGGNWFDP | Homotypic G1 |
| 46 | | | 4-59*01 | 3-9*01 | 2*01 | K | 3-20*01 | 4*01 | ARNFPSYTPDWFFDL | Homotypic G1 |
| 57 | | | 3-30*01 | 6-19*01 | 4*02 | L | 1-40*01 | J1*01 | ARAAGIMVAGTFLTEFYFDY | Heterotypic G1, G2, G3 |

^{A}, mAB is heterotypic but with low minimum neutralization concentraion
ND, VP5* or VP8* specificity not determined

TABLE 4

| RV strain | G serotype | P genotype | Strain origin |
|---|---|---|---|
| Wa | G1 | P[8] | Human |
| CDC-9 | G1 | P[8] | Human |
| DS1 | G2 | P[4] | Human |
| ST3 | G4 | P[6] | Human |
| 69M | G8 | P[10] | Human |
| 116E | G9 | P[11] | Human |
| #321 | G10 | P[11] | Human |
| WI61 | G9 | P[8] | Human |
| L26 | G12 | P[4] | Human |
| RRV | G4 | P[3] | Simian |
| D x RRV | G1 | P[3] | Mono VP7 reassortants |
| DS1 x RRV | G3 | P[3] | Mono VP7 reassortants |
| ST3 x RRV | G4 | P[3] | Mono VP7 reassortants |
| OSU | G5 | P[7] | Porcine |
| NCDV | G6 | P[1] | Bovine |
| UK | G9 | P[5] | Bovine |

TABLE 5

| | | | | | | | | | | | | | | | | | Immuno- |
|---|---|---|---|---|---|---|---|---|---|---|---|---|---|---|---|---|---|
| | | | | | | | | | | | | | | | | | precipitation |
| | | | | | | | | | | | | | | Binding to | | | of |
| | | | | | | | | | Binding to BV-expressed | | | | | recombinant | | | RV |
| | | | Endpoint immunostaining concentration | | | | | | viral proteins expressed | | | | | RV VP8* proteins | | | VP5* |
| | RV | | (ng/ml) of RV-infected cells | | | | | | in S19 cells | | | | | by ELISA | | | protein |
| Mab | protein | | Wa | DS1 | ST3 | RRV | NCDV | OSU | VP4 | VP4 | VP4 | VP7 | VP6 | VP8* | VP8* | VP8* | VP5* |
| No. | specificity | | G1P[8] | G2P[4] | G4P[6] | G3P[3] | G6P[1] | G5[7] | P[8] | P[6] | P[4] | G1 | RRV | [P8] | [P8] | [P8] | [P3] |
| 2 | VP4 | VP5* | 5.0 | 5.0 | — | — | — | — | + | + | + | — | — | — | — | — | + |
| 30 | | VP5* | 5.0 | 5.0 | 5.0 | | 50.0 | 6.6 | + | + | + | — | — | — | — | — | + |
| 32 | | VP5* | 50.0 | 50.0 | 5.0 | 50.0 | 50.0 | 5.0 | + | + | + | — | — | — | — | — | + |
| 41 | | VP5* | 0.5 | 5.0 | 0.5 | 5.0 | 5.6 | 5.6 | + | + | + | — | — | — | — | — | + |
| 49 | | VP5* | 5.0 | 0.5 | 5.0 | 0.5 | 5.6 | 5.6 | + | + | + | — | — | — | — | — | + |
| 4 | | VP8* | 0.5 | 0.5 | — | — | — | — | + | — | + | — | — | + | + | + | |
| 6 | | VP8* | 0.5 | 0.5 | — | — | — | — | + | — | + | — | — | + | — | + | |
| 8 | | VP8* | 5.0 | 50.0 | — | — | — | — | + | — | + | — | — | + | — | + | |
| 9 | | VP8* | 0.5 | 0.5 | — | — | — | — | + | — | + | — | — | + | + | + | |
| 11 | | VP8* | 0.5 | 500.0 | — | — | — | — | + | — | + | — | — | + | — | — | |
| 12 | | VP8* | 5.0 | 50.0 | — | — | — | — | + | — | + | — | — | + | — | + | |
| 13 | | VP8* | 5.0 | — | — | — | — | — | + | — | — | — | — | + | — | — | |
| 14 | | VP8* | 5.0 | 5.0 | — | — | — | — | + | — | + | — | — | + | — | + | |
| 15 | | VP8* | 0.5 | 5.0 | — | — | — | — | + | — | + | — | — | + | — | + | |
| 16 | | VP8* | 0.5 | 0.5 | — | — | — | — | + | — | + | — | — | + | — | + | |
| 18 | | VP8* | 5.0 | 5.0 | 50.0 | — | — | — | + | — | + | — | — | + | + | + | |
| 19 | | VP8* | 5.0 | 5.0 | — | — | — | — | + | — | + | — | — | + | — | + | |
| 20 | | VP8* | 5.0 | 5.0 | — | — | — | — | + | — | + | — | — | + | — | + | |
| 21 | | VP8* | 0.5 | 5.0 | — | — | — | — | + | — | + | — | — | + | — | + | |
| 23 | | VP8* | 5.0 | 5.0 | — | — | — | — | — | — | + | — | — | + | — | + | |
| 29 | | VP8* | 5.0 | — | — | — | — | — | + | — | — | — | — | + | — | — | |
| 31 | | VP8* | 5.0 | 50.0 | — | — | — | — | + | — | + | — | — | + | — | — | |
| 35 | | VP8* | 5.0 | 5.0 | — | — | 5.0 | 50.0 | + | — | + | — | — | + | — | + | |
| 43 | | VP8* | 5.0 | — | — | — | — | — | + | — | — | — | — | + | — | — | |
| 44 | | VP8* | 0.5 | 50.0 | — | — | — | — | + | — | — | — | — | + | — | + | |
| 55 | | VP8* | 5.0 | 0.5 | 5.0 | — | — | — | + | — | + | — | — | + | + | + | |
| 56 | | VP8* | 5.0 | — | 500.0 | 500.0 | 500.0 | — | + | — | — | — | — | + | — | — | |
| 47 | | ND | 0.5 | 0.5 | — | — | — | — | + | — | + | — | — | — | — | — | |
| 62 | | ND | 0.5 | 0.5 | — | — | — | — | + | — | + | — | — | — | — | — | |
| 22 | VP7 | | 500.0 | — | — | — | — | — | — | — | — | + | — | — | — | — | |
| 27 | | | 0.5 | — | — | — | — | — | — | — | — | + | — | — | — | — | |
| 46 | | | 0.5 | — | — | — | — | — | — | — | — | + | — | — | — | — | |
| 57 | | | 0.5 | 0.5 | 500.0 | 5.0 | — | 50.0 | — | — | — | + | — | — | — | — | |
| 10 | VP6 | | 0.5 | 5.0 | 5.0 | 5.0 | 0.5 | 0.5 | — | — | — | — | + | — | — | — | |

### REFERENCES

Madhi, S. A., et al., Effect of human rotavirus vaccine on severe diarrhea in African infants. N Engl J Med, 2010. 362(4): p. 289-98.
Armah, G. E., et al., Efficacy of pentavalent rotavirus vaccine against severe rotavirus gastroenteritis in infants in developing countries in sub-Saharan Africa: a randomised, double-blind, placebo-controlled trial. Lancet, 2010. 376(9741): p. 606-14.
Zaman, K., et al., Efficacy of pentavalent rotavirus vaccine against severe rotavirus gastroenteritis in infants in developing countries in Asia: a randomised, double-blind, placebo-controlled trial. Lancet, 2010. 376(9741): p. 615-23.
Linhares, A. C., et al., Efficacy and safety of an oral live attenuated human rotavirus vaccine against rotavirus gastroenteritis during the first 2 years of life in Latin American infants: a randomised, double-blind, placebo-controlled phase III study. Lancet, 2008. 371(9619): p. 1181-9.
Boom, J. A., et al., Effectiveness of pentavalent rotavirus vaccine in a large urban population in the United States. Pediatrics, 2010. 125(2): p. e199-207.
Buttery, J. P., et al., Reduction in rotavirus-associated acute gastroenteritis following introduction of rotavirus vaccine into Australia's National Childhood vaccine schedule. Pediatr Infect Dis J, 2011. 30(1 Suppl): p. S25-9.
Vesikari, T., et al., Safety and efficacy of a pentavalent human-bovine (WC3) reassortant rotavirus vaccine. N Engl J Med, 2006. 354(1): p. 23-33.
Patton, J. T., et al., Coupling of rotavirus genome replication and capsid assembly. Adv Virus Res, 2007. 69: p. 167-201.
Prasad, B. V., et al., Three-dimensional structure of rotavirus. J Mol Biol, 1988. 199(2): p. 269-75.
Fiore, L., H. B. Greenberg, and E. R. Mackow, The VP8 fragment of VP4 is the rhesus rotavirus hemagglutinin. Virology, 1991. 181(2): p. 553-63.
Denisova, E., et al., Rotavirus capsid protein VP5* permeabilizes membranes. J Virol, 1999. 73(4): p. 3147-53.
Fiore, L., et al., Antigenicity, immunogenicity and passive protection induced by immunization of mice with baculovirus-expressed VP7 protein from rhesus rotavirus. J Gen Virol, 1995. 76 (Pt 8): p. 1981-8.
Greenberg, H. B., et al., Production and preliminary characterization of monoclonal antibodies directed at two surface proteins of rhesus rotavirus. J Virol, 1983. 47(2): p. 267-75.
Greenberg, H. B., et al., Rescue of noncultivatable human rotavirus by gene reassortment during mixed infection with ts mutants of a cultivatable bovine rotavirus. Proc Natl Acad Sci USA, 1981. 78(1): p. 420-4.
Kalica, A. R., J. Flores, and H. B. Greenberg, Identification of the rotaviral gene that codes for hemagglutination and protease-enhanced plaque formation. Virology, 1983. 125(1): p. 194-205.
Mackow, E. R., et al., The rhesus rotavirus gene encoding protein VP3: location of amino acids involved in homologous and heterologous rotavirus neutralization and identification of a putative fusion region. Proc Natl Acad Sci USA, 1988. 85(3): p. 645-9.
Matsui, S. M., et al., Passive protection against rotavirus-induced diarrhea by monoclonal antibodies to the heterotypic neutralization domain of VP7 and the VP8 fragment of VP4. J Clin Microbiol, 1989. 27(4): p. 780-2.
Offit, P. A., et al., Reassortant rotaviruses containing structural proteins vp3 and vp7 from different parents induce antibodies protective against each parental serotype. J Virol, 1986. 60(2): p. 491-6.
Taniguchi, K., et al., Antibody response to serotype-specific and cross-reactive neutralization epitopes on VP4 and VP7 after rotavirus infection or vaccination. J Clin Microbiol, 1991. 29(3): p. 483-7.
Aoki, S. T., et al., Structure of rotavirus outer-layer protein VP7 bound with a neutralizing Fab. Science, 2009. 324(5933): p. 1444-7.
Dormitzer, P. R., et al., Structural rearrangements in the membrane penetration protein of a non-enveloped virus. Nature, 2004. 430(7003): p. 1053-8.
Zhang, X., et al., Evidence for a role of the 5-HT1B receptor and its adaptor protein, p11, in L-DOPA treatment of an animal model of Parkinsonism. Proc Natl Acad Sci USA, 2008. 105(6): p. 2163-8.
Velazquez, F. R., et al., Rotavirus infections in infants as protection against subsequent infections. N Engl J Med, 1996. 335(14): p. 1022-8.
Ruiz-Palacios, G. M., et al., Safety and efficacy of an attenuated vaccine against severe rotavirus gastroenteritis. N Engl J Med, 2006. 354(1): p. 11-22.
Bhandari, N., After eradication: India's post-polio problem. BMJ, 2014. 348: p. g2275.
Higo-Moriguchi, K., et al., Isolation of human monoclonal antibodies that neutralize human rotavirus. J Virol, 2004. 78(7): p. 3325-32.
Lu, D. R., et al., Identifying functional anti-Staphylococcus aureus antibodies by sequencing antibody repertoires of patient plasmablasts. Clin Immunol, 2014. 152(1-2): p. 77-89.
Tan, Y. C., et al., Barcode-enabled sequencing of plasmablast antibody repertoires in rheumatoid arthritis. Arthritis Rheumatol, 2014. 66(10): p. 2706-15.
Georgiou, G., et al., The promise and challenge of high-throughput sequencing of the antibody repertoire. Nat Biotechnol, 2014. 32(2): p. 158-68.
Tian, C., et al., Immunodominance of the VH1-46 antibody gene segment in the primary repertoire of human rotavirus-specific B cells is reduced in the memory compartment through somatic mutation of nondominant clones. Journal of immunology, 2008. 180(5): p. 3279-88.
Weitkamp, J. H., et al., Infant and adult human B cell responses to rotavirus share common immunodominant variable gene repertoires. Journal of immunology, 2003. 171(9): p. 4680-8.
Ward, R. L., et al., Immunodominance of the VP4 neutralization protein of rotavirus in protective natural infections of young children. J Virol, 1993. 67(1): p. 464-8.
Di Niro, R., et al., Rapid generation of rotavirus-specific human monoclonal antibodies from small-intestinal mucosa. J Immunol, 2010. 185(9): p. 5377-83.
Nair, N., et al., High-dimensional immune profiling of total and rotavirus VP6-specific intestinal and circulating B cells by mass cytometry. Mucosal Immunol, 2015.
Narvaez, C. F., et al., Human rotavirus-specific IgM Memory B cells have differential cloning efficiencies and switch capacities and play a role in antiviral immunity in vivo. J Virol, 2012. 86(19): p. 10829-40.
Gladstone, B. P., et al., Protective effect of natural rotavirus infection in an Indian birth cohort. N Engl J Med, 2011. 365(4): p. 337-46.
Ward, R. L. and M. M. McNeal, VP6: A candidate rotavirus vaccine. J Infect Dis, 2010. 202 Suppl: p. S101-7.
Burns, J. W., et al., Protective effect of rotavirus VP6-specific IgA monoclonal antibodies that lack neutralizing activity. Science, 1996. 272(5258): p. 104-7.
Feng, N., et al., Inhibition of rotavirus replication by a non-neutralizing, rotavirus VP6-specific IgA mAb. J Clin Invest, 2002. 109(9): p. 1203-13.
Vega, C. G., et al., Recombinant monovalent llama-derived antibody fragments (VHH) to rotavirus VP6 protect neonatal gnotobiotic piglets against human rotavirus-induced diarrhea. PLoS pathogens, 2013. 9(5): p. e1003334.
   Aiyegbo, M. S., et al., Human rotavirus VP6-specific antibodies mediate intracellular neutralization by binding to a quaternary structure in the transcriptional pore. PLoS One, 2013. 8(5): p. e61101.
   Cuadras, M. A., et al., Gene expression pattern in Caco-2 cells following rotavirus infection. J Virol, 2002. 76(9): p. 4467-82.
   Bridger, J. C. and G. N. Woode, Characterization of two particle types of calf rotavirus. J Gen Virol, 1976. 31(2): p. 245-50.
   Cohen, J., Ribonucleic acid polymerase activity associated with purified calf rotavirus. J Gen Virol, 1977. 36(3): p. 395-402.
   Charpilienne, A., et al., Individual rotavirus-like particles containing 120 molecules of fluorescent protein are visible in living cells. J Biol Chem, 2001. 276(31): p. 29361-7.
   Carreno-Torres, J. J., et al., Characterization of viroplasm formation during the early stages of rotavirus infection. Virol J, 2010. 7: p. 350.
   Ishida, S., et al., Quantification of systemic and local immune responses to individual rotavirus proteins during rotavirus infection in mice. J Clin Microbiol, 1996. 34(7): p. 1694-700.
   Hoshino, Y., et al., Serotypic similarity and diversity of rotaviruses of mammalian and avian origin as studied by plaque-reduction neutralization. J Infect Dis, 1984. 149(5): p. 694-702.

1. An isolated antibody that specifically binds to a rotavirus protein, and comprises a set of CDR sequences derived from any one of SEQ ID NO: 19 and 20; SEQ ID NO:21 and 22; SEQ ID NO:23 and 24; SEQ ID NO:25 and 26; SEQ ID NO:27 and 28; SEQ ID NO:29 and 30; SEQ ID NO:31 and 32; SEQ ID NO:33 and 34; and SEQ ID NO:35 and 36; and an antibody that competes for binding therewith.

2. The isolated antibody of claim 1, wherein the antibody comprises intact variable regions from any one of SEQ ID NO: 19 and 20; SEQ ID NO:21 and 22; SEQ ID NO:23 and 24; SEQ ID NO:25 and 26; SEQ ID NO:27 and 28; SEQ ID NO:29 and 30; SEQ ID NO:31 and 32; SEQ ID NO:33 and 34; and SEQ ID NO:35 and 36.

3. The antibody of claim 1, wherein the antibody is a human monoclonal antibody.

4. The antibody of claim 1, wherein the antibody is a variable region fragment.

5. The antibody of claim 4, wherein the antibody is a single chain variable region fragment.

6. The antibody of claim 1, wherein the antibody is other than an IgA antibody.

7. The antibody of claim 1, wherein the antibody neutralizes rotavirus in an in vitro assay.

8. The antibody of claim 1, wherein the antibody inhibits rotavirus infection in vivo in a subject.

9. The antibody of claim 1, wherein the antibody binds to two or more serotypes of a rotavirus protein or fragment thereof.

10. A polynucleotide encoding an antibody set forth in claim 1.

11. A cell that produces an antibody set forth in claim 1.

12. A pharmaceutical composition comprising an effective dose of an antibody set forth in claim 1.

13. A method of treating rotavirus infection in a mammal comprising, administering to the mammal an antibody as set forth in claim 1 or a formulation of claim 12, such that infection of cells by rotavirus is inhibited.

14. A method of detecting a rotavirus infection in a mammal comprising, contacting a body fluid of a mammal with an antibody as set forth in claim 1, and determining if binding occurs, said binding being indicative of the presence of a rotavirus infection.

15. A screening method for determining whether a rotavirus antigen of interest produces a heterotypic antibody response, comprising:
immunizing an individual with a candidate rotavirus immunogen;
sorting contacting cells of intestinal RV-specific IgA⁺ antibody secreting cells with triple-layered RV particles conjugated to a detectable label
sorting single cells by flow cytometry;
sequencing antibody coding sequences produced by the sorted single cells;
determining whether the sequenced antibodies neutralize multiple serotypes; wherein a rotavirus antigen that produces a heterotypic antibody response generates antibodies that neutralize multiple serotypes.


SEQUENCE LISTING (36 sequences)

SEQ ID NO: 1 - DNA, 478 bp, Homo sapiens
cggcgcctacctgagatcaccggtgaattcgccaccatgtacaggatgcaactcctgtcttgcattgcactaagtcttgcacttgtcacaaacagtgaggtgcagctggtggagtctggaggaggattggtaaagcctggaggatcccttagactctcctgtaaagcctccggactcattgtcagtgacgcatggatgagctgggtccgccagtctccaggaaagggactggagtgggttggacgtattaaaagcgaaattaatggtggaacaatagactacgctgcaccagtgaaaggaagattcacaatcttaagagatgattcaaagaacactctgtatctgcaaatcaacagcctgaaaaccgaggacacagccgtatattactgtaccacgcgactgctgttctctccatggggacagggaaccctggtcaccgtctcctcagctagcaccaagggcccatcggtcttccccc

SEQ ID NO: 2 - DNA, 467 bp, Homo sapiens
tgtgaccggcgcctacctgagatcaccggtgccaccatgtacaggatgcaactcctgtcttgcattgcactaagtcttgcacttgtcacaaacagtcagcctgtgctgactcagccaccttcttcctccgcatctcctggagaatccgcaagactcacctgcaccttgcccagtgacatcaatgttgcttactacaacatatactggtaccagcagaagccaggaagccctcccaggtatctcctctactactactcagactcagatcagggacagggatctggagtccccagccgattctctggatccaaggatgcttcagcaaatacgggaattttattcatctcaggactccagtctgaggatgaggctgactattattgtatgatttggacaagcaatgcttcaatgttcggaggaggaaccaagttgaccgtcctaggtcagcccaaggctgccccctcggt

SEQ ID NO: 3 - DNA, 500 bp, Homo sapiens
cggcgcctacctgagatcaccggtgaattcgccaccatgtacaggatgcaactcctgtcttgcattgcactaagtcttgcacttgtcacaaacagtcaggtgcagctgcaggagtcgggcccaggactggtcaagcctttggagaccctgtccctcacctgcgctgtctctggtgtctccatcaatagttactactggagctggatccggcagccaccaggaaagggactggagtggattggcaatgtcttttatagtgggagcaccaagtacaatccctccctcgagagtcgagtcgccatgacagttgactcgtccaggaatcaggtctccctgaggctgaactctgtgaccgctgcggacacggccgtgtattactgtgcgagagaaggagtgggatacggctacaataattacggaggtaactggttcgacccctggggacagggaactctggtcaccgtctcgtcagctagcaccaagggcccatcggtcttccc

SEQ ID NO: 4 - DNA, 450 bp, Homo sapiens
tgtgaccggcgcctacctgagatcaccggtgccaccatgtacaggatgcaactcctgtcttgcattgcactaagtcttgcacttgtcacaaacagtgaagttgtgttgacgcagtctccaggcaccctgtctttgtctccaggagaaagagtcaccctctcttgtagggccagtcagagtgttaccagtagtaacttagcctggtaccagcagaaacctggacagactcccaggctcctcatttctggtgcatccagcagggccactggaatcccagacaggttcagtggaagtggatctgggacagacttcactctcaccatcagcagactggagcctgaagattttgcagtgtattactgtcagcaatatgctaactcacccgtcactttcggaggaggaaccaagctggagatcaaacgtacggtggctgcaccatctgtcttcatc

SEQ ID NO: 5 - DNA, 496 bp, Homo sapiens
cggcgcctacctgagatcaccggtgaattcgccaccatgtacaggatgcaactcctgtcttgcattgcactaagtcttgcacttgtcacaaacagtcaggtgcagctggtgcagtctggagctgaggtgaagaagcctggagcctcagtgacagtttcctgcaaggcatctggatacgccttcaccagtttctatctacactgggtgcgacaggcccctggacaaggacttgagtggatgggaataatcaaccctagtgatggtcgcacaagatacgcacagaagttccagggaagagtcaccatgaccagcgacacgtccacgaacacagtctacgtggagctgagcagcctgagatctgaggacacggccatatattactgtgcgagaggtgccatcgggaactacaatgcccgggaggctttggacgtctggggacgaggaaccaccgtcaccgtctcctcagctagcaccaagggcccatcggtcttccccc

SEQ ID NO: 6 - DNA, 453 bp, Homo sapiens
tgtgaccggcgcctacctgagatcaccggtgccaccatgtacaggatgcaactcctgtcttgcattgcactaagtcttgcacttgtcacaaacagtgaaatagtgatgacgcagtctccagccaccctgtctgtgtctccaggggaaagtgccaccctctcctgcagggccagtcagagtattaacagtaacttagcctggtaccagcagaagcctggccaggctcccaggctcctcatttttagtgcatcctccagggccactggtatcccagccagattcagtggcagtgggtctgggacagagttcacactcaccatcagcagcctgcagtctgacgattttgcagtttattactgtcagcagtataatatttggcctccggagcacacttttggccaggggaccaggctgcagatcaaacgtacggtggctgcaccatctgtcttcatc

SEQ ID NO: 7 - DNA, 508 bp, Homo sapiens
cggcgcctacctgagatcaccggtgaattcgccaccatgtacaggatgcaactcctgtcttgcattgcactaagtcttgcacttgtcacaaacagtgacgtgcagctggtggagtctggaggaggattggtccagcctggaggtccatcgagactctcctgttcagcctctagattcaccttcagtaattatgctatgtactgggtccgccaggctccaggaaagggactggaatatgtttcatctattagtagtgatggaggtagcacatattacgcagagtccgtgaagggcagattcaccatctccagagacaattccaagaacacactgtatcttcaaatgaggagtctgagagctgaggacgccgctgtgtattactgtgtgacagatgtcttgaggttaccctatagcactggctggagcccaggagactttatctactggggacagggaaccctggtcaccgtctcctcagctagcaccaagggcccatcggtcttccccc

SEQ ID NO: 8 - DNA, 444 bp, Homo sapiens
tgtgaccggcgcctacctgagatcaccggtgccaccatgtacaggatgcaactcctgtcttgcattgcactaagtcttgcacttgtcacaaacagtgacatccagatgacccagtctccttccatcctgtatgcatctgtaggagacagagtcaccatcacttgccgagcaagtcagagtgttagcagctggttggcctggtatcagcagaaaccagggaaagtccctaaactcctcatctatcaggcgtctactttagaaaatggagtcccatcaagattcagcggaagtggatctggaacagaattcattctcaccatcagcagcctgcagcctgatgattttgcaacttattactgccaacattataatgttttgtggacgttcggacaaggaaccaaggtggaaatcaagcgtacggtggctgcaccatctgtcttcatc

SEQ ID NO: 9 - DNA, 498 bp, Homo sapiens
cggcgcctacctgagatcaccggtgaattcgccaccatgtacaggatgcaactcctgtcttgcattgcactaagtcttgcacttgtcacaaacagtgaggtgcagctggtggagtctggaggaggaccagtacagcctggaggatccctgaaactctcctgtgcagcctctggattcaccttcagtaattatgaaatgtactgggtccgccaggctccaggaaagggactggagtgggtttcatacattagtactagtccagctatcacatattatgcagactctgtgaggggacgattcaccatctccagagacaacgccaagagctcactgtatctgcacatgaacagcctgagagccgaggacacggctgtttactactgtgcgaccatttcccaccaacaatttagcagtggctggaacgcctggttcgacccatggggacagggaaccctggtcaccgtctcctcagctagcaccaagggcccatcggtcttc

SEQ ID NO: 10 - DNA, 455 bp, Homo sapiens
tgtgaccggcgcctacctgagatcaccggtgccaccatgtacaggatgcaactcctgtcttgcattgcactaagtcttgcacttgtcacaaacagtaactttatgctgactcaaccccactctgtgtcggagtctccaggaaagacggtaaccatctcctgcaccggaagcagtggcagcattgccagcaactatgtgcagtggtaccggcagcgaccaggaagtgcaccaaccactgtgatctatgaaaattaccaaagaccatctggagtccctgctcgattctctggatccatcgacaggtcctccaactctgcctccctcaccatctctggactgcagactgacgacgaggctgactactactgtcagtcttatgacaacaacaatctttgggtgttcggaggaggaaccaagctgaccgtcctaggtcagcccaaggctgccccctcggt

SEQ ID NO: 11 - DNA, 363 bp, Homo sapiens
caggtgcagctgcaggagtcgggcccaggactggtgaagccttcggagaccctgtccctcacgtgcactgtctctggtggctccatcaatagttactactggagttggatccggcagtccccagggaagggactggagtggattgggtatgtcttttacagtggcatcaccaagtacaacccctccctccagagtcgagtcaccatatcactggacatgggcaagaaccagttctccctgaagttgacctctgtgaatgctgcggacgcggccgtgtattattgtgcgagaaactttccgagctacaccccggactggttttttgatctctggggccgtggcaccctggtcactgtctcctca

SEQ ID NO: 12 - DNA, 324 bp, Homo sapiens
gaaattgtgttgacgcagtctccaggcaccctgtctttgtctccaggggaaagagccaccctctcctgcagggccagtcagagtgttagtagtgataacttagcctggtaccagcagaaacctggccagcctcccaggctcctcatctatggtgcatcccacagggccactggcatcccagacaggttcagtggcagtgggtctggaacagacttcactctcaccatcagcagactggagcctgaagatttcgcagtttatcactgtcagcagtatggtagctcaccgctcactttcggcggagggaccaaggtggagatcaaa

SEQ ID NO: 13 - DNA, 390 bp, Homo sapiens
caggtgcagctgcaggagtcgggcccaggactggtgaagccctcggagaccctgtccctcacctgctctgtctctggtggctccatcagtgtttactactggaactggatccggcagtccccagggaagggactggagtggattgcgtctatgtattacactggaattaccaactacaacccctccctcaagagtcgagtcaccatgtcagtagacatgtccaagaaccagttctccctgaagctgagctctgtgaccgctgcggacacggccgtgtattactgtgcgagaacgatggggattgaccagaataaccgtggctggccccctgcgggctactacttcggcatggacgtctggggccaagggaccacggtcaccgtctcctca

SEQ ID NO: 14 - DNA, 336 bp, Homo sapiens
gatattgtgatgactcagtccccactctccctgcccgtcacacctggagagccggcctccatctcctgcaggtctagtcagagcctcctgcatagtaatggaaacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttgggatttattactgcatgcaagctctagaagcctcgctcactttcggcggagggaccaaggtggagatcaag

SEQ ID NO: 15 - DNA, 381 bp, Homo sapiens
gacgtgcagctggtggagtctgggggaggcttggtccagcctggggggtccgtgagactctcctgttcagcctctagattcaccttcagtaattatgctatgtactgggtccgccaggctccagggaagggactggaatatgtttcatctattagtagtgatgggggtagcacatattacgcagagtccgtgaagggcagattcaccatctccagagacaattccaagaacacactgtatcttcaaatgaggagtctgagagctgaggacgccgctgtgtattactgtgtgacagatgtcttgaggttaccctatagcactggctggagcccaggggactttatctactggggccagggaaccctggtcaccgtctcctca

SEQ ID NO: 16 - DNA, 318 bp, Homo sapiens
gacatccagatgacccagtctccttccatcctgtatgcatctgtaggagacagagtcaccatcacttgccgggccagtcagagtgttagcagctggttggcctggtatcagcagaaaccagggaaagtccctaaactcctcatctatcaggcgtctactttagaaaatggggtcccatcaagattcagcggcagtggatctgggacagaattcattctcaccatcagcagcctgcagcctgatgattttgcaacttattactgccaacattataatgttttgtggacgttcggccaagggaccaaggtggaaatcaag

SEQ ID NO: 17 - DNA, 378 bp, Homo sapiens
caggtgcagttggtggagtctgggggaggcgtggtccagtctgggaggtccctgagactctcctgtgcagcctctggattcaccttcagaagctatgctatgcactgggtccgccaggctccaggcaaggggctagagtgggtggcagatttatcattagatggaagtcataaatacgcagactccgtgaggggccgattcaccatctccagcgacagttccaagaacacggtgtatctgcaaatgaacagcctgagaactgaggacacggctatatattactgtgcgagagccgcgggtataatggtggctggtactttcttaaccgagttctactttgactactggggccagggaaccctggtcaccgtctcctca

SEQ ID NO: 18 - DNA, 336 bp, Homo sapiens
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacatcaagcggccctcaggggtccctgaccgattctctggctccaagtctggcacctcagcctccctggccatcactgggctccagactgaggatgaggctgactattactgccagtcctatgacagcagcctgagtgcctattatgtcttcggaactgggaccagggtcaccgtccta

SEQ ID NO: 19 - protein, 120 aa, Homo sapiens
IGHEVQLVESGGGLVKPGGSLRLSCKASGLIVSDAWMSWVRQSPGKGLEWVGRIKSEINGGTIDYAAPVKGRFTILRDDSKNTLYLQINSLKTEDTAVYYCTTRLLFSPWGQGTLVTVSS

SEQ ID NO: 20 - protein, 115 aa, Homo sapiens
QPVLTQPPSSSASPGESARLTCTLPSDINVAYYNIYWYQQKPGSPPRYLLYYYSDSDQGQGSGVPSRFSGSKDASANTGILFISGLQSEDEADYYCMIWTSNASMFGGGTKLTVL

SEQ ID NO: 21 - protein, 128 aa, Homo sapiens
IGHQVQLQESGPGLVKPLETLSLTCAVSGVSINSYYWSWIRQPPGKGLEWIGNVFYSGSTKYNPSLESRVAMTVDSSRNQVSLRLNSVTAADTAVYYCAREGVGYGYNNYGGNWFDPWGQGTLVTVSS

SEQ ID NO: 22 - protein, 108 aa, Homo sapiens
EVVLTQSPGTLSLSPGERVTLSCRASQSVTSSNLAWYQQKPGQTPRLLISGASSRATGIPDRFSGSGSGTDFTLTISRLEPEDFAVYYCQQYANSPVTFGGGTKLEIK

SEQ ID NO: 23 - protein, 126 aa, Homo sapiens
IGHQVQLVQSGAEVKKPGASVTVSCKASGYAFTSFYLHWVRQAPGQGLEWMGIINPSDGRTRYAQKFQGRVTMTSDTSTNTVYVELSSLRSEDTAIYYCARGAIGNYNAREALDVWGRGTTVTVSS

SEQ ID NO: 24 - protein, 109 aa, Homo sapiens
EIVMTQSPATLSVSPGESATLSCRASQSINSNLAWYQQKPGQAPRLLIFSASSRATGIPARFSGSGSGTEFTLTISSLQSDDFAVYYCQQYNIWPPEHTFGQGTRLQIK

SEQ ID NO: 25 - protein, 130 aa, Homo sapiens
IGHDVQLVESGGGLVQPGGPSRLSCSASRFTFSNYAMYWVRQAPGKGLEYVSSISSDGGSTYYAESVKGRFTISRDNSKNTLYLQMRSLRAEDAAVYYCVTDVLRLPYSTGWSPGDFIYWGQGTLVTVSS

SEQ ID NO: 26 - protein, 105 aa, Homo sapiens
DIQMTQSPSILYASVGDRVTITCRASQSVSSWLAWYQQKPGKVPKLLIYQASTLENGVPSRFSGSGSGTEFILTISSLQPDDFATYYCQHYNVLWTFGQGTKVEI

SEQ ID NO: 27 - protein, 128 aa, Homo sapiens
IGHEVQLVESGGGPVQPGGSLKLSCAASGFTFSNYEMYWVRQAPGKGLEWVSYISTSPAITYYADSVRGRFTISRDNAKSSLYLHMNSLRAEDTAVYYCATISHQQFSSGWNAWFDPWGQGTLVTVSS

SEQ ID NO: 28 - protein, 111 aa, Homo sapiens
NFMLTQPHSVSESPGKTVTISCTGSSGSIASNYVQWYRQRPGSAPTTVIYENYQRPSGVPARFSGSIDRSSNSASLTISGLQTDDEADYYCQSYDNNNLWVFGGGTKLTVL

SEQ ID NO: 29 - protein, 121 aa, Homo sapiens
QVQLQESGPGLVKPSETLSLTCTVSGGSINSYYWSWIRQSPGKGLEWIGYVFYSGITKYNPSLQSRVTISLDMGKNQFSLKLTSVNAADAAVYYCARNFPSYTPDWFFDLWGRGTLVTVSS

SEQ ID NO: 30 - protein, 108 aa, Homo sapiens
EIVLTQSPGTLSLSPGERATLSCRASQSVSSDNLAWYQQKPGQPPRLLIYGASHRATGIPDRFSGSGSGTDFTLTISRLEPEDFAVYHCQQYGSSPLTFGGGTKVEIK

SEQ ID NO: 31 - protein, 130 aa, Homo sapiens
QVQLQESGPGLVKPSETLSLTCSVSGGSISVYYWNWIRQSPGKGLEWIASMYYTGITNYNPSLKSRVTMSVDMSKNQFSLKLSSVTAADTAVYYCARTMGIDQNNRGWPPAGYYFGMDVWGQGTTVTVSS

SEQ ID NO: 32 - protein, 112 aa, Homo sapiens
DIVMTQSPLSLPVTPGEPASISCRSSQSLLHSNGNNYLDWYLQKPGQSPQLLIYLGSNRASGVPDRFSGSGSGTDFTLKISRVEAEDVGIYYCMQALEASLTFGGGTKVEIK

SEQ ID NO: 33 - protein, 127 aa, Homo sapiens
DVQLVESGGGLVQPGGSVRLSCSASRFTFSNYAMYWVRQAPGKGLEYVSSISSDGGSTYYAESVKGRFTISRDNSKNTLYLQMRSLRAEDAAVYYCVTDVLRLPYSTGWSPGDFIYWGQGTLVTVSS

SEQ ID NO: 34 - protein, 106 aa, Homo sapiens
DIQMTQSPSILYASVGDRVTITCRASQSVSSWLAWYQQKPGKVPKLLIYQASTLENGVPSRFSGSGSGTEFILTISSLQPDDFATYYCQHYNVLWTFGQGTKVEIK

SEQ ID NO: 35 - protein, 126 aa, Homo sapiens
QVQLVESGGGVVQSGRSLRLSCAASGFTFRSYAMHWVRQAPGKGLEWVADLSLDGSHKYADSVRGRFTISSDSSKNTVYLQMNSLRTEDTAIYYCARAAGIMVAGTFLTEFYFDYWGQGTLVTVSS

SEQ ID NO: 36 - protein, 112 aa, Homo sapiens
QSVLTQPPSVSGAPGQRVTISCTGSSSNIGAGYDVHWYQQLPGTAPKLLIYGNIKRPSGVPDRFSGSKSGTSASLAITGLQTEDEADYYCQSYDSSLSAYYVFGTGTRVTVL